SEQUENCE LISTING (15 sequences)

SEQ ID NO: 1 - DNA, 1632 bp, Homo sapiens
atgggtcctggggcgcggctggcggcgctgctggcggtgctggcgctcgggacaggagacccagaaagggctgcggctcggggcgacacgttctcggcgctgaccagcgtggcgcgcgccctggcgcccgagcgccggctgctggggctgctgaggcggtacctgcgcggggaggaggcgcggctgcgggacctgactagattctacgacaaggtactttctttgcatgaggattcaacaacccctgtggctaaccctctgcttgcatttactctcatcaaacgcctgcagtctgactggaggaatgtggtacatagtctggaggccagtgagaacatccgagctctgaaggatggctatgagaaggtggagcaagaccttccagcctttgaggaccttgagggagcagcaagggccctgatgcggctgcaggacgtgtacatgctcaatgtgaaaggcctggcccgaggtgtctttcagagagtcactggctctgccatcactgacctgtacagccccaaacggctcttttctctcacaggggatgactgcttccaagttggcaaggtggcctatgacatgggggattattaccatgccattccatggctggaggaggctgtcagtctcttccgaggatcttacggagagtggaagacagaggatgaggcaagtctagaagatgccttggatcacttggcctttgcttatttccgggcaggaaatgtttcgtgtgccctcagcctctctcgggagtttcttctctacagcccagataataagaggatggccaggaatgtcttgaaatatgaaaggctcttggcagagagccccaaccacgtggtagctgaggctgtcatccagaggcccaatataccccacctgcagaccagagacacctacgaggggctatgtcagaccctgggttcccagcccactctctaccagatccctagcctctactgttcctatgagaccaattccaacgcctacctgctgctccagcccatccggaaggaggtcatccacctggagccctacattgctctctaccatgacttcgtcagtgactcagaggctcagaaaattagagaacttgcagaaccatggctacagaggtcagtggtggcatcaggggagaagcagttacaagtggagtaccgcatcagcaaaagtgcctggctgaaggacactgttgacccaaaactggtgaccctcaaccaccgcattgctgccctcacaggccttgatgtccggcctccctatgcagagtatctgcaggtggtgaactatggcatcggaggacactatgagcctcactttgaccatgctacgtcaccaagcagccccctctacagaatgaagtcaggaaaccgagttgcaacatttatgatctatctgagctcggtggaagctggaggagccacagccttcatctatgccaacctcagcgtgcctgtggttaggaatgcagcactgttttggtggaacctgcacaggagtggtgaaggggacagtgacacacttcatgctggctgtcctgtcctggtgggagataagtgggtggccaacaagtggatacatgagtatggacaggaattccgcagaccctgcagctccagccctgaagac

SEQ ID NO: 2 - protein, 543 aa, Homo sapiens
GPGARLAALLAVLALGTGDPERAAARGDTFSALTSVARALAPERRLLGLLRRYLRGEEARLRDLTRFYDKVLSLHEDSTTPVANPLLAFTLIKRLQSDWRNVVHSLEASENIRALKDGYEKVEQDLPAFEDLEGAARALMRLQDVYMLNVKGLARGVFQRVTGSAITDLYSPKRLFSLTGDDCFQVGKVAYDMGDYYHAIPWLEEAVSLFRGSYGEWKTEDEASLEDALDHLAFAYFRAGNVSCALSLSREFLLYSPDNKRMARNVLKYERLLAESPNHVVAEAVIQRPNIPHLQTRDTYEGLCQTLGSQPTLYQIPSLYCSYETNSNAYLLLQPIRKEVIHLEPYIALYHDFVSDSEAQKIRELAEPWLQRSVVASGEKQLQVEYRISKSAWLKDTVDPKLVTLNHRIAALTGLDVRPPYAEYLQVVNYGIGGHYEPHFDHATSPSSPLYRMKSGNRVATFMIYLSSVEAGGATAFIYANLSVPVVRNAALFWWNLHRSGEGDSDTLHAGCPVLVGDKWVANKWIHEYGQEFRRPCSSSPED

SEQ ID NO: 3 - DNA, 1509 bp, Homo sapiens
atggcggcagcggcggtgacaggccagcggcctgagaccgcggcggccgaggaggcctcgaggccgcagtgggcgccgccagaccactgccaggctcaggcggcggccgggctgggcgacggcgaggacgcaccggtgcgtccgctgtgcaagccccgcggcatctgctcgcgcgcctacttcctggtgctgatggtgttcgtgcacctgtacctgggtaacgtgctggcgctgctgctcttcgtgcactacagcaacggcgacgaaagcagcgatcccgggccccaacaccgtgcccagggccccgggcccgagcccaccttaggtcccctcacccggctggagggcatcaaggtggggcacgagcgtaaggtccagctggtcaccgacagggatcacttcatccgaaccctcagcctcaagccgctgctcttcgaaatccccggcttcctgactgatgaagagtgtcggctcatcatccatctggcgcagatgaaggggttacagcgcagccagatcctgcctactgaagagtatgaagaggcaatgagcactatgcaggtcagccagctggacctcttccggctgctggaccagaaccgtgatgggcaccttcagctccgtgaggttctggcccagactcgcctgggaaatggatggtggatgactccagagagcattcaggagatgtacgccgcgatcaaggctgaccctgatgctgacggagtgctgagtctgcaggagttctccaacatggaccttcgggacttccacaagtacatgaggagccacaaggcagagtccagtgagctggtgcggaacagccaccatacctggctctaccagggtgagggtgcccaccacatcatgcgtgccatccgccagagggtgctgcgcctcactcgcctgtcgcctgagatcgtggagctcagcgagccgctgcaggttgttcgatatggtgaggggggccactaccatgcccacgtggacagtgggcctgtgtacccagagaccatctgctcccataccaagctggtagccaacgagtctgtacccttcgagacctcctgccgctacatgacagtgctgttttatttgaacaacgtcactggtgggggcgagactgttttccctgtagcagataacagaacctacgatgaaatgagtctgattcaggatgacgtggacctccgtgacacacggaggcactgtgacaagggaaacctgcgtgtcaagccccaacagggcacagcagtcttctggtacaactacctgcctgatgggcaaggttgggtgggtgacgtagacgactactcgctgcacgggggctgcctggtcacgcgcggcaccaagtggattgccaacaactggattaatgtggaccccagccgagcgcggcaagcgctgttccaacaggagatggcccgccttgcccgagaagggggcaccgactcacagcccgagtgggctctggaccgggcctaccgcgatgcgcgcgtggaactctga

SEQ ID NO: 4 - protein, 502 aa, Homo sapiens [sequence omitted]

SEQ ID NO: 5 - DNA, 19 bp, Homo sapiens
agcctcctggaagaaggcc

SEQ ID NO: 6 - DNA, 21 bp, Homo sapiens
ggtaacaacctctcccttgcc

SEQ ID NO: 7 - DNA, 24 bp, Homo sapiens
tgtcagctttgtctgtgcctcgca

SEQ ID NO: 8 - DNA, 21 bp, Homo sapiens
gcagactaaaggtctggccaa

SEQ ID NO: 9 - DNA, 20 bp, Homo sapiens
ataggaactgcgccgtatcg

SEQ ID NO: 10 - DNA, 18 bp, Homo sapiens
tcttgccccaccccgcca

SEQ ID NO: 11 - DNA, 24 bp, Artificial Sequence
  random oligonucleotide
tcaactgactagatgtacatggac

SEQ ID NO: 12 - DNA, 172 bp, Artificial Sequence
  HIF reporter construct consisting of a minimal promoter containing a tanem of hpoxia responsible elemens and the CMV promoter TATA box
tacgcgtgctagcccgggctcgatgccagactccacagtgcatacgtgggctccaacaggtcctcttccctcccagtcagatcctccacagtgcatacgtgggctccaacaggtcctcttaggtctatataagcagaagcttggcattccggtactgttggtaaagccacca

SEQ ID NO: 13 - DNA, 2248 bp, Homo sapiens
gaagttcgcgagcgctggcatgggtcctggggcgcggctggcggcgctgctggcggtgctggcgctcgggacaggagacccagaaagggctgcggctcggggcgacacgttctcggcgctgaccagcgtggcgcgcgccctggcgcccgagcgccggctgctggggctgctgaggcggtacctgcgcggggaggaggcgcggctgcgggacctgactagattctacgacaaggtactttctttgcatgaggattcaacaacccctgtggctaaccctctgcttgcatttactctcatcaaacgcctgcagtctgactggaggaatgtggtacatagtctggaggccagtgagaacatccgagctctgaaggatggctatgagaaggtggagcaagaccttccagcctttgaggaccttgagggagcagcaagggccctgatgcggctgcaggacgtgtacatgctcaatgtgaaaggcctggcccgaggtgtctttcagagagtcactggctctgccatcactgacctgtacagccccaaacggctcttttctctcacaggggatgactgcttccaagttggcaaggtggcctatgacatgggggattattaccatgccattccatggctggaggaggctgtcagtctcttccgaggatcttacggagagtggaagacagaggatgaggcaagtctagaagatgccttggatcacttggcctttgcttatttccgggcaggaaatgtttcgtgtgccctcagcctctctcgggagtttcttctctacagcccagataataagaggatggccaggaatgtcttgaaatatgaaaggctcttggcagagagccccaaccacgtggtagctgaggctgtcatccagaggcccaatataccccacctgcagaccagagacacctacgaggggctatgtcagaccctgggttcccagcccactctctaccagatccctagcctctactgttcctatgagaccaattccaacgcctacctgctgctccagcccatccggaaggaggtcatccacctggagccctacattgctctctaccatgacttcgtcagtgactcagaggctcagaaaattagagaacttgcagaaccatggctacagaggtcagtggtggcatcaggggagaagcagttacaagtggagtaccgcatcagcaaaagtgcctggctgaaggacactgttgacccaaaactggtgaccctcaaccaccgcattgctgccctcacaggccttgatgtccggcctccctatgcagagtatctgcaggtggtgaactatggcatcggaggacactatgagcctcactttgaccatgctacgtcaccaagcagccccctctacagaatgaagtcaggaaaccgagttgcaacatttatgatctatctgagctcggtggaagctggaggagccacagccttcatctatgccaacctcagcgtgcctgtggttaggaatgcagcactgttttggtggaacctgcacaggagtggtgaaggggacagtgacacacttcatgctggctgtcctgtcctggtgggagataagtgggtggccaacaagtggatacatgagtatggacaggaattccgcagaccctgcagctccagccctgaagactgaactgttggcagagagaagctggtggagtcctgtggctttccagagaagccaggagccaaaagctggggtaggagaggagaaagcagagcagcctcctggaagaaggccttgtcagctttgtctgtgcctcgcaaatcagaggcaagggagaggttgttaccaggggacactgagaatgtacatttgatctgccccagccacggaagtcagagtaggatgcacagtacaaaggaggggggagtggaggcctgagagggaagtttctggagttcagatactctctgttgggaacaggacatctcaacagtctcaggttcgatcagtgggtcttttggcactttgaaccttgaccacagggaccaagaagtggcaatgaggacacctgcaggaggggctagcctgactcccagaactttaagactttctccccactgccttctgctgcagcccaagcagggagtgtccccctcccagaagcatatcccagatgagtggtacattatataaggattttttttaagttgaaaacaactttcttttctttttgtatgatggttttttaacacagtcattaaaaatgtttataaatcaaaa

SEQ ID NO: 14 - DNA, 1820 bp, Homo sapiens
tgccatggcggcagcggcggtgacaggccagcggcctgagaccgcggcggccgaggaggcctcgaggccgcagtgggcgccgccagaccactgccaggctcaggcggcggccgggctgggcgacggcgaggacgcaccggtgcgtccgctgtgcaagccccgcggcatctgctcgcgcgcctacttcctggtgctgatggtgttcgtgcacctgtacctgggtaacgtgctggcgctgctgctcttcgtgcactacagcaacggcgacgaaagcagcgatcccgggccccaacaccgtgcccagggccccgggcccgagcccaccttaggtcccctcacccggctggagggcatcaaggtggggcacgagcgtaaggtccagctggtcaccgacagggatcacttcatccgaaccctcagcctcaagccgctgctcttcgaaatccccggcttcctgactgatgaagagtgtcggctcatcatccatctggcgcagatgaaggggttacagcgcagccagatcctgcctactgaagagtatgaagaggcaatgagcactatgcaggtcagccagctggacctcttccggctgctggaccagaaccgtgatgggcaccttcagctccgtgaggttctggcccagactcgcctgggaaatggatggtggatgactccagagagcattcaggagatgtacgccgcgatcaaggctgaccctgatgctgacggagtgctgagtctgcaggagttctccaacatggaccttcgggacttccacaagtacatgaggagccacaaggcagagtccagtgagctggtgcggaacagccaccatacctggctctaccagggtgagggtgcccaccacatcatgcgtgccatccgccagagggtgctgcgcctcactcgcctgtcgcctgagatcgtggagctcagcgagccgctgcaggttgttcgatatggtgaggggggccactaccatgcccacgtggacagtgggcctgtgtacccagagaccatctgctcccataccaagctggtagccaacgagtctgtacccttcgagacctcctgccgctacatgacagtgctgttttatttgaacaacgtcactggtgggggcgagactgttttccctgtagcagataacagaacctacgatgaaatgagtctgattcaggatgacgtggacctccgtgacacacggaggcactgtgacaagggaaacctgcgtgtcaagccccaacagggcacagcagtcttctggtacaactacctgcctgatgggcaaggttgggtgggtgacgtagacgactactcgctgcacgggggctgcctggtcacgcgcggcaccaagtggattgccaacaactggattaatgtggaccccagccgagcgcggcaagcgctgttccaacaggagatggcccgccttgcccgagaagggggcaccgactcacagcccgagtgggctctggaccgggcctaccgcgatgcgcgcgtggaactctgagggaagagttagccccggttcccagccgcgggtcgccagttgcccaagatcaggggtccggctgtccttctgtcctgctgcagactaaaggtctggccaatgtcttgccccaccccgccagccgcgatacggcgcagttcctatattcatgttatttattgtgtactgactccatctgccccgtcaaataaaaaaccacaaggttcgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRQYFPNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLSRATVHDPETGKLTTAQYRVSKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

(19) United States
(12) Patent Application Publication
(10) Pub. No.: US 2006/0110769 A1
(43) Pub. Date: May 25, 2006

(54) Regulation of novel human prolyl 4-hydroxylases

(75) Inventors: Shyam Ramakrishnan, Brighton, MA (US); Ingo Flamme, Reichshof (DE); Felix Oehme, Wuppertal (DE)

Correspondence Address: BANNER & WITCOFF, 1001 G STREET N W, SUITE 1100, WASHINGTON, DC, 20001, US

(73) Assignee: Bayer Aktiengesellschaft, Leverkusen (DE)

(21) Appl. No.: 11/316,907

(22) Filed: Dec. 27, 2005

Related U.S. Application Data
(62) Division of application No. 10/135,843, filed on May 1, 2002.
(60) Provisional application No. 60/287,715, filed on May 2, 2001.
(60) Provisional application No. 60/372,110, filed on Apr. 15, 2002.

(51) Int. Cl. C12Q 1/68; C12Q 1/30

(52) U.S. Cl. 435/6; 435/27

(57) ABSTRACT

Reagents that regulate human prolyl 4-hydroxylase and reagents which bind to human prolyl 4-hydroxylase gene products can play a role in preventing, ameliorating, or correcting dysfunctions or diseases including, but not limited to cardiovascular disorders, cancer, inflammatory diseases, fibrotic disorders, anemia, and CNS disorders, such as stroke.

This application claims the benefit of and incorporates by reference co-pending provisional application Ser. No. 60/287,715 filed May 2, 2001 and Ser. No. 60/372,110 filed Apr. 15, 2002.

### FIELD OF THE INVENTION

The invention relates to novel human prolyl 4-hydroxylases and their regulation for the treatment of disease are disclosed.

### BACKGROUND OF THE INVENTION

Prolyl 4-hydroxylases comprise a family of enzymes that are involved in posttranslational modification of a variety of proteins. The prolyl 4-hydroxylation of procollagen has been analyzed in he most detail. Hydroxylation of proline residues is a prerequisite for the folding of the newly synthesized procollagen polypeptide chain into its typical triple helical structure. Active prolyl 4-hydroxylases are tetramers of 2 alpha and 2 beta subunits. The known beta subunit is identical to the enzyme protein disulfide isomerase (PDI). The catalytic activity of the enzyme probably resides in the carboxy-terminal half of the alpha subunit. Kivirikko & Myllyharju, Matrix Biol. 16, 357-68, 1998. Prolyl 4-hydroxylation of collagen is of crucial importance for any pathological process that is related to overproduction of collagen, such as fibrotic alterations of liver, heart, lung, and skin. Modulation of human prolyl 4-hydroxylases can be useful for the therapy of diseases characterized by fibrotic alterations. Franklin, Int. J. Biochem. Cell. Biol. 29, 79-89, 1997.

Prolyl 4-hydroxylation of certain nuclear factors also is implicated in the regulation of oxygen-dependent gene expression. The regulation of tissue oxygen supply is of crucial importance for all processes in human life. The level of tissue oxygenation results from the balance between oxygen supply and oxygen consumption. This balance is exactly tuned in the healthy organism but is disturbed under many pathological conditions such as pulmonary and cardiovascular diseases, which are characterized by a decrease in oxygen supply, as well as cancer and inflammations, which both are characterized by an increased demand of oxygen within the diseased tissue.

In addition to immediate physiological responses such as vasodilatation, adaptation of heart rate, etc., imbalance of tissue oxygenation is followed by modulation of the transcription rate of a multitude of genes. Among these genes are those that encode for important growth factors and hormones (e.g., vascular endothelial growth factor and erythropoietin) and many metabolic enzymes. The transcriptional modulation leads, for example, to a long lasting adaptation of metabolism, growth, or regression of blood vessels and increased or decreased erythropoiesis.

All oxygen-regulated genes are target genes for a distinct family of nuclear transcription factors which are termed hypoxia inducible factors (HIFs). The oxygen-regulated genes carry distinct binding sites for HIFs in their regulatory elements (i.e., promoters and enhancers). Wenger & Gassmann, Biol. Chem. 378, 609-16, 1997; Semenza, Ann. Rev. Cell. Dev. Biol. 15, 551-78, 1999; Zhu & Bunn, Respir. Physiol. 115, 239-47, 1999. In their active form, hypoxia inducible factors consist of an alpha and a beta subunit. While the beta subunit, which is named HIF-1beta or ARNT, is not regulated in response to changes of tissue oxygen, the alpha subunit is unstable under normoxic or hyperoxic conditions. This is due to the rapid degradation of the constitutively translated alpha subunit via the proteasomal pathway. The alpha subunit becomes ubiquitinylated via an E3 ubiquitin conjugase complex, in which the VHL tumor suppressor protein is the central adaptor protein to the alpha subunit. Ohh et al., Nat. Cell. Biol. 2, 423-27, 2000; Kondo & Kaelin, Exp. Cell Res. 264, 117-25, 2001.

The ubiquitin conjugase complex can only bind to the alpha subunit and initiate degradation if the alpha subunit is hydroxylated on a distinct proline residue. This residue is highly conserved among HIFs. Under hypoxic conditions (low tissue oxygen), prolyl 4-hydroxylation of this residue does not take place, and HIFs become stable and can activate their target genes. The prolyl 4-hydroxylase(s) involved in prolyl 4-hydroxylation of HIF-alpha have not been identified. Ivan et al., Science 292, 64-68, 2001; Jaakkola et al., Science 292, 468-72, 2001. Thus, any HIF-alpha specific prolyl 4-hydroxylase is a key oxygen sensor for the regulation of oxygen sensitive genes, such as vascular endothelial growth factor, erythropoietin, and iNOS and therefore is of crucial importance for cardiovascular, neoplastic, and inflammatory diseases.

There is a need in the art to identify additional prolyl 4-hydroxylases, which can be regulated to treat cancers, anemias, chronic inflammatory diseases, and cardiovascular diseases.

### BRIEF SUMMARY OF THE INVENTION

One embodiment of the invention is an isolated and purified protein comprising a first polypeptide segment comprising the amino acid sequence shown in SEQ ID NO:2 or SEQ ID NO:4.

Another embodiment of the invention is an isolated and purified protein comprising an amino acid sequence which differs from the amino acid sequences shown in SEQ ID NO:2 or SEQ ID NO:4 by between one and ten conservative amino acid substitutions and which has a prolyl 4-hydroxylase activity.

Yet another embodiment of the invention is an isolated and purified polypeptide comprising a first polypeptide segment which comprises between 9 and 543 contiguous amino acids of a human prolyl 4-hydroxylase protein as shown in SEQ ID NO:2.

Still another embodiment of the invention is an isolated and purified polypeptide comprising a first polypeptide segment which comprises between 9 and 502 contiguous amino acids of a human prolyl 4-hydroxylase protein as shown in SEQ ID NO:4.

Even another embodiment of the invention is a purified preparation of antibodies which specifically bind to a human prolyl 4-hydroxylase protein comprising an amino acid sequence shown in SEQ ID NO:2 or SEQ ID NO:4.

A further embodiment of the invention is an isolated and purified polynucleotide which encodes the amino acid sequence shown in SEQ ID NO:2 or SEQ ID NO:4.

Yet another embodiment of the invention is a cDNA molecule which encodes the amino acid sequence shown in SEQ ID NO:2 or SEQ ID NO:4.

Another embodiment of the invention is a cDNA molecule which encodes a portion of the amino acid sequence shown in SEQ ID NO:2 or SEQ ID NO:4.

Still another embodiment of the invention is an isolated and purified single-stranded probe comprising between 8 and 1629 contiguous nucleotides of a coding sequence for a human prolyl 4-hydroxylase protein or the complement thereof, wherein the prolyl 4-hydroxylase protein comprises the amino acid sequence shown in SEQ ID NO:2.

Even another embodiment of the invention is an isolated and purified single-stranded probe comprising between 8 and 1506 contiguous nucleotides of a coding sequence for a human prolyl 4-hydroxylase protein or the complement thereof, wherein the prolyl 4-hydroxylase protein comprises the amino acid sequence shown in SEQ ID NO:4.

Yet another embodiment of the invention is an isolated and purified antisense oligonucleotide comprising a first sequence of between 8 and 1632 contiguous nucleotides which is complementary to a second sequence of between 8 and 1632 contiguous nucleotides found in a coding sequence for a human prolyl 4-hydroxylase protein which comprises the amino acid sequence shown in SEQ ID NO:2.

A further embodiment of the invention is an isolated and purified antisense oligonucleotide comprising a first sequence of between 8 and 1509 contiguous nucleotides which is complementary to a second sequence of between 8 and 1509 contiguous nucleotides found in a coding sequence for a human prolyl 4-hydroxylase protein which comprises the amino acid sequence shown in SEQ ID NO:4.

Still another embodiment of the invention is a container comprising a set of primers. The set comprises a first primer and a second primer. The first primer comprises at least 8 contiguous nucleotides which is complementary to a contiguous sequence of nucleotides located at the 5′ end of the coding strand of a double-stranded polynucleotide which encodes a human prolyl 4-hydroxylase protein as shown in SEQ ID NO:2 or SEQ ID NO:4. The second primer comprises at least 8 contiguous nucleotides which is complementary to a contiguous sequence of nucleotides located at the 5′ end of the non-coding strand of the polynucleotide.

A further embodiment of the invention is an expression vector comprising a coding sequence for a human prolyl 4-hydroxylase protein comprising the amino acid sequence shown in SEQ ID NO:2 or SEQ ID NO:4 and a promoter which is located upstream from the coding sequence and which controls expression of the coding sequence.

Another embodiment of the invention is a host cell comprising an expression vector. The expression vector comprises a coding sequence for a human prolyl 4-hydroxylase protein comprising the amino acid sequence shown in SEQ ID NO:2 or SEQ ID NO:4 and a promoter which is located upstream from the coding sequence and which controls expression of the coding sequence.

Still another embodiment of the invention is a method of producing a human prolyl 4-hydroxylase protein. A host cell is cultured in a culture medium. The host cell comprises an expression construct comprising (a) a coding sequence for a human prolyl 4-hydroxylase protein comprising the amino acid sequence shown in SEQ ID NO:2 or SEQ ID NO:4 and (b) a promoter which is located upstream from the coding sequence and which controls expression of the coding sequence. The step of culturing is carried out under conditions whereby the protein is expressed. The protein is recovered from the culture medium.

Even another embodiment of the invention is a method of detecting a human prolyl 4-hydroxylase expression product. A test sample is contacted with a reagent that specifically binds to an expression product of a prolyl 4-hydroxylase coding sequence as shown in SEQ ID NO:1 or SEQ ID NO:3. The test sample is assayed to detect binding between the reagent and the expression product. The test sample is identified as containing a human prolyl 4-hydroxylase expression product if binding between the reagent and the expression product is detected.

Yet another embodiment of the invention is a method of treating. An effective amount of a reagent that either (a) decreases expression of a gene that encodes a human prolyl 4-hydroxylase protein comprising the amino acid sequence shown in SEQ ID NO:2 or SEQ ID NO:4 or (b) decreases effective levels of the prolyl 4-hydroxylase protein is administered to a patient with cancer, an inflammatory disorder, or a fibrotic disease. Symptoms of the cancer, inflammatory disorder, or fibrotic disease are thereby reduced.

A further embodiment of the invention is a method of treating. An effective amount of a prolyl 4-hydroxylase protein comprising the amino acid sequence shown in SEQ ID NO:2 or SEQ ID NO:4, an agonist of the prolyl 4-hydroxylase protein, or an expression vector encoding the prolyl 4-hydroxylase protein is administered to a patient with a cardiovascular disease, anemia, or a stroke. Symptoms of the cardiovascular disease, anemia, or stroke are thereby reduced.

Another embodiment of the invention is a method of screening for candidate therapeutic agents that may be useful for treating cancer, an inflammatory disorder, or a fibrotic disease. A human prolyl 4-hydroxylase protein comprising the amino acid sequence shown in SEQ ID NO:2 or SEQ ID NO:4 is contacted with a test compound. Binding between the prolyl 4-hydroxylase protein and the test compound is assayed. A test compound that binds to the prolyl 4-hydroxylase protein is identified as a candidate therapeutic agent that may be useful for treating cancer, an inflammatory disorder, or a fibrotic disease.

Yet another embodiment of the invention is a method of screening for candidate therapeutic agents. Expression of a polynucleotide encoding a human prolyl 4-hydroxylase protein comprising the amino acid sequence of SEQ ID NO:2 or SEQ ID NO:4 is assayed in the presence and absence of a test compound. A test compound that decreases the expression is identified as a candidate therapeutic agent that may be useful for treating cancer, an inflammatory disorder, or a fibrotic disease. A test compound that increases the expression is identified as a candidate therapeutic agent for treating cardiovascular disorders, anemia, or stroke.

Still another embodiment of the invention is a pharmaceutical composition comprising a reagent which binds to an expression product of a gene which encodes a prolyl 4-hydroxylase protein comprising the amino acid sequence shown in SEQ ID NO:2 or SEQ ID NO:4 and a pharmaceutically acceptable carrier.

Yet another embodiment of the invention is a pharmaceutical composition comprising a human prolyl 4-hydroxylase protein comprising the amino acid sequence shown in SEQ ID NO:2 or SEQ ID NO:4 and a pharmaceutically acceptable carrier.

A further embodiment of the invention is a pharmaceutical composition comprising a polynucleotide encoding a human prolyl 4-hydroxylase protein comprising the amino acid sequence shown in SEQ ID NO:2 or SEQ ID NO:4 and a pharmaceutically acceptable carrier.

The invention thus provides novel human prolyl 4-hydroxylase proteins, reagents that modulate the activity of the proteins, and diagnostic and therapeutic methods.

### BRIEF DESCRIPTION OF THE FIGURES

FIG. 1. BLASTP—alignment of PH-1 (SEQ ID NO:2) against gb|AAA59069.1| (U14620) alpha-subunit of prolyl 4-hydroxylase [Homo sapiens] procollagen-proline dioxygenase (EC 1.14.11.2) alpha chain precursor (SEQ ID NO:15), splice form 1-human. Length=534. Score=312 bits (791), Expect=7e-84. Identities=185/477 (38%), Positives=273/477 (56%), Gaps=41/477 (8%).

FIG. 2. BLASTP—alignment of PH-2 (SEQ ID NO:4) against gb|AAA59069.1|. (U14620) alpha-subunit of prolyl 4-hydroxylase [Homo sapiens] procollagen-proline dioxygenase (EC 1.14.11.2) alpha chain precursor (SEQ ID NO:15), splice form 1-human. Length=534. Score=76.1 bits (184), Expect=1e-12. Identities=62/201 (30%), Positives=80/201 (38%), Gaps=53/201 (26%).

FIG. 3. Expression of PH-1 in human tissues as analyzed by quantitative PCR techniques (TaqMan).

FIG. 4 Expression of PH-2 in human tissues as analyzed by quantitative PCR techniques (TaqMan).

FIG. 5. Sequence (SEQ ID NO:12) present in HIF-RE2 luc, HIF reporter construct constructed in pGL3 (Promega), consisting of a minimal promoter containing a tandem of hypoxia responsible elements (bold underlined) and the CMV promoter TATA box (underlined).

FIG. 6. Activity of double mutant HIF-1 alpha. FIG. 6A, cotransfected with EGLN3. FIG. 6B, cotransfected with PH-2.

FIG. 7. Activity of HIF-2 alpha in the presence of PH-2 and EGLN3 (upper panel) and disappearance of HIF-2 alpha protein in the presence of PH-2 and EGLN3 (lower panel).

### DETAILED DESCRIPTION OF THE INVENTION

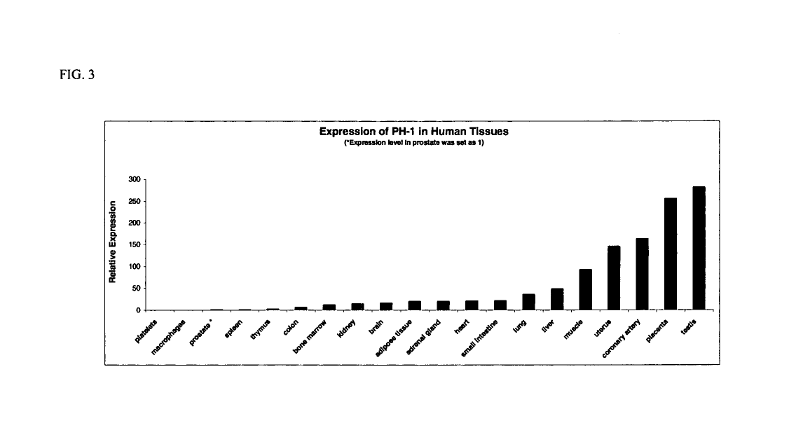

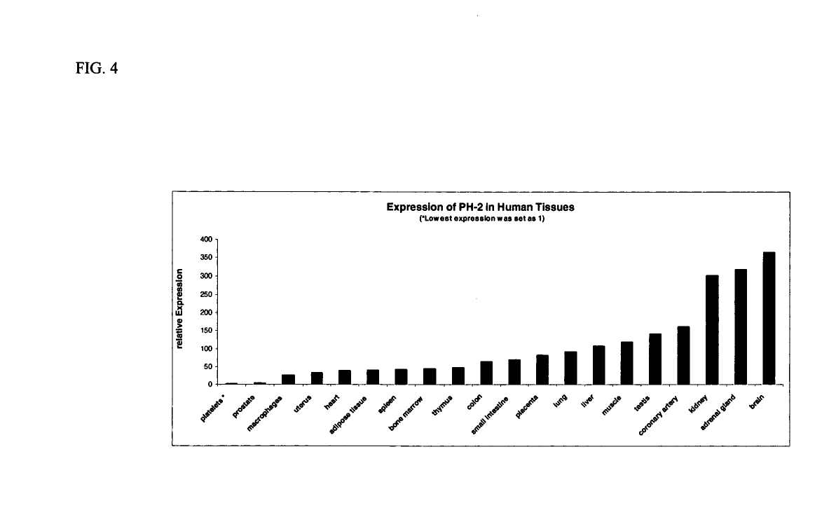

Two novel human prolyl 4-hydroxylases, referred to hereafter as PH-1 and PH-2, are a discovery of the present invention. Prolyl 4-hydroxylases (EC 1.14.11.2) are endoplasmic-reticulum bound dioxygenases which form a tetrameric complex (alpha 2 beta 2) with protein disulfide isomerase (EC 5.3.4.1). Two isoforms of prolyl 4-hydroxylase (alpha I and alpha II) are currently known. These enzymes catalyze the hydroxylation of -X-Pro-Gly-sequences in collagen and collagen-like proteins, which is essential for the correct folding of collagen peptide chains at body temperature. Prolyl 4-hydroxylases have been identified in several species (e.g., human, mouse, rat, and chicken). Expression patterns of PH-1 and PH-2 are shown in FIGS. 3 and 4.

Hydroxylation of proline by prolyl 4-hydroxylase requires 2-oxoglutarate, O₂, Fe²⁺ and ascorbate. The following are examples of prolyl 4-hydroxylase reactions:
1) Procollagen L-proline+2-oxoglutarate+O₂<->procollagen trans-4-hydroxy-L-proline+succinate+CO₂
2) (Pro-Pro-Gly)ₓ+2-oxoglutarate+O₂<->(Pro-Pro-OH-Gly)ₓ+succinate+CO₂
   where x=1-10.

The C-terminal catalytic domain of prolyl 4-hydroxylases, which binds Fe²⁺ and 2-oxoglutarate, is highly conserved. Site-directed mutagenesis reveals that His₄₂₉, Asp₄₃₁, and His₅₀₀ of the alpha I subunit are essential for Fe²⁺ binding and enzyme activity. Lys₅₁₀ was shown to bind the C-5 carboxyl group of 2-oxoglutarate. His₅₁₈ is another critical residue that is involved in the correct orientation of the C-1 carboxyl group of 2-oxoglutarate.

A peptide binding domain consisting of ˜100 amino acid residues is located in the N-terminal region of prolyl 4-hydroxylases. Mutations of Ile₁₉₉ and Tyr₂₅₀ in the alpha I subunit abolish the binding of prolyl 4-hydroxylase to poly(L-proline), a competitive inhibitor of the type I enzyme. These two residues are, however, not highly conserved but can be replaced by other amino acid residues, for example in the type II enzyme, which reflects the different binding specificities of the alpha I and alpha II isoform for proline-rich peptide substrates. Numbering of amino acid residues is that shown in gb|AAA59069.1|(U14620), alpha-subunit of prolyl 4-hydroxylase [Homo sapiens] (SEQ ID NO:15).

“Human prolyl 4-hydroxylase” as used herein refers to either PH-1, PH-2, or both. Human prolyl 4-hydroxylase PH-1 comprises the amino acid sequence shown in SEQ ID NO:2. A coding sequence for human prolyl 4-hydroxylase is shown in SEQ ID NO:1. Human prolyl 4-hydroxylase PH-2 comprises the amino acid sequence shown in SEQ ID NO:4. A coding sequence for human prolyl 4-hydroxylase is shown in SEQ ID NO:3. These sequences are located within the longer sequences shown in SEQ ID NOS:13 and 14, respectively.

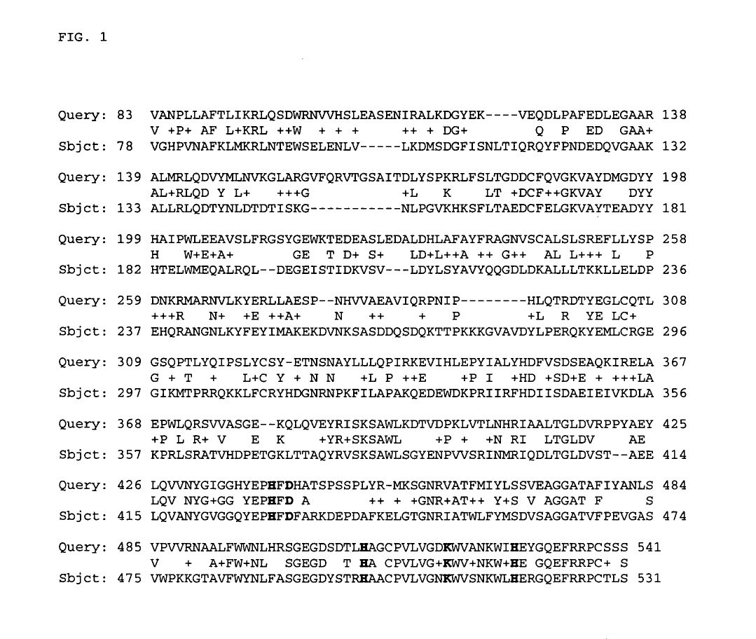

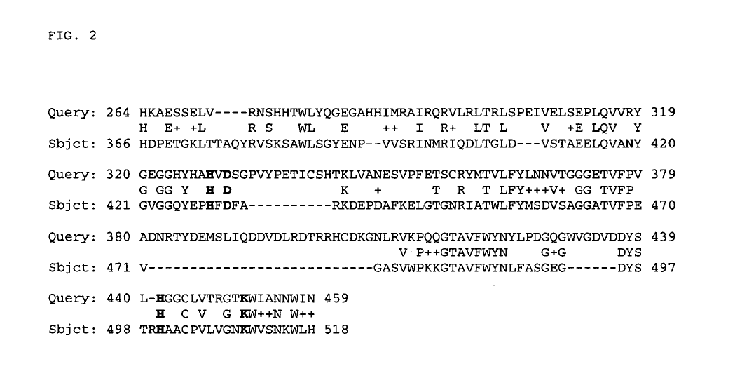

BLAST searches identified the oxoglutarate and Fe²⁺ binding domain in SEQ ID NO:2 and SEQ ID NO:4, which also is the catalytic domain. BLAST alignments show 38% and 30% identity to human prolyl 4-Hydroxylase alpha(I) for PH-1 and PH-2, respectively (FIGS. 1 and 2). The conserved amino acid residues (shown in FIGS. 1 and 2 in bold) are involved in the catalytic function of all known prolyl 4-hydroxylases. Kivirikko & Myllyhaiju, Matrix Biol. 16, 357-68, 1998.

Human prolyl 4-hydroxylases PH-1 and PH-2 of the invention are useful in therapeutic methods to treat disorders such as cancer, cardiovascular disorders, anemia, CNS disorders, inflammatory diseases, and fibrotic disorders. PH-1 and PH-2 also can be used to screen for human prolyl 4-hydroxylase activators and inhibitors.

Polypeptides

Human prolyl 4-hydroxylase polypeptides according to the invention comprise at least 6, 10, 15, 20, 25, 50, 75, 100, 125, 150, 175, 200, 225, 250, 275, 300, 325, 350, 375, 400, 425, 450, 475, 500, 525, or 543 contiguous amino acids selected from the amino acid sequence shown in SEQ ID NO:2 or a biologically active variant thereof, as defined below, or at least 6, 10, 15, 20, 25, 50, 75, 100, 125, 150, 175, 200, 225, 250, 275, 300, 325, 350, 375, 400, 425, 450, 475, 500, or 502 contiguous amino acids selected from the amino acid sequence shown in SEQ ID NO:4 or a biologically active variant thereof, as defined below. A prolyl 4-hydroxylase polypeptide of the invention therefore can be a portion of a prolyl 4-hydroxylase protein, a full-length prolyl 4-hydroxylase protein, or a fusion protein comprising all or a portion of a prolyl 4-hydroxylase protein.

Biologically Active Variants

Human prolyl 4-hydroxylase polypeptide variants which are biologically active, e.g., retain a prolyl 4-hydroxylase activity, also are human prolyl 4-hydroxylase polypeptides. Preferably, naturally or non-naturally occurring human prolyl 4-hydroxylase polypeptide variants have amino acid sequences which are at least about 39, 40, 45, 50, 55, 60, 65, or 70, preferably about 75, 80, 85, 90, 95, 96, 97, 98, or 99% identical to the amino acid sequence shown in SEQ ID NO:2 or a fragment thereof or at least 31, 35, 40, 45, 50, 55, 60, 65, or 70, preferably about 75, 80, 85, 90, 95, 96, 97, 98, or 99% identical to the amino acid sequence shown in SEQ ID NO:4 or a fragment thereof. Percent identity between a putative human prolyl 4-hydroxylase polypeptide variant and an amino acid sequence of SEQ ID NO:2 or 4 is determined by conventional methods. See, for example, Altschul et al., Bull. Math. Bio. 48:603 (1986), and Henikoff & Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff & Henikoff, 1992.

Those skilled in the art appreciate that there are many established algorithms available to align two amino acid sequences. The “FASTA” similarity search algorithm of Pearson & Lipman is a suitable protein alignment method for examining the level of identity shared by an amino acid sequence disclosed herein and the amino acid sequence of a putative variant. The FASTA algorithm is described by Pearson & Lipman, Proc. Nat'l Acad. Sci. USA 85:2444(1988), and by Pearson, Meth. Enzymol. 183:63 (1990). Briefly, FASTA first characterizes sequence similarity by identifying regions shared by the query sequence (e.g., SEQ ID NO:2 or 4) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman & Wunsch, J. Mol. Biol.48:444 (1970); Sellers, SIAM J. Appl. Math.26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine. Polypeptide variants can comprise between one and ten or more conservative amino acid substitutions relative to SEQ ID NO:2 or 4 (e.g., 1, 2, 3, 4, 5, 6, 7, 8, 9, or 10 substitutions).

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a human prolyl 4-hydroxylase polypeptide can be found using computer programs well known in the art, such as DNASTAR software.

The invention additionally, encompasses prolyl 4-hydroxylase polypeptides that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications can be carried out by known techniques including, but not limited, to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The prolyl 4-hydroxylase polypeptides may also be modified with a detectable label, such as an enzymatic, fluorescent, isotopic or affinity label to allow for detection and isolation of the protein.

The invention also provides chemically modified derivatives of prolyl 4-hydroxylase polypeptides that may provide additional advantages such as increased solubility, stability and circulating time of the polypeptide, or decreased immunogenicity (see U.S. Pat. No. 4,179,337). The chemical moieties for derivitization can be selected from water soluble polymers such as polyethylene glycol, ethylene glycol/propylene glycol copolymers, carboxymethylcellulose, dextran, polyvinyl alcohol, and the like. The polypeptides can be modified at random or predetermined positions within the molecule and can include one, two, three, or more attached chemical moieties.

Whether an amino acid change or a polypeptide modification results in a biologically active prolyl 4-hydroxylase polypeptide can readily be determined by assaying for prolyl 4-hydroxylase activity, as described for example, in Kivirikko & Myllylä, Methods Enzymol. 82, 245-304, 1982, or Cuncliffe et al., Biochem. J. 240, 617-19, 1986.

Fusion Proteins

Fusion proteins are useful for generating antibodies against prolyl 4-hydroxylase polypeptide amino acid sequences and for use in various assay systems. For example, fusion proteins can be used to identify proteins that interact with portions of a prolyl 4-hydroxylase polypeptide. Protein affinity chromatography or library-based assays for protein-protein interactions, such as the yeast two-hybrid or phage display systems, can be used for this purpose. Such methods are well known in the art and also can be used as drug screens.

A prolyl 4-hydroxylase polypeptide fusion protein comprises two polypeptide segments fused together by means of a peptide bond. The first polypeptide segment comprises at least 6, 10, 15, 20, 25, 50, 75, 100, 125, 150, 175, 200, 225, 250, 275, 300, 325, 350, 375, 400, 425, 450, 475, 500, 525, or 543 contiguous amino acids selected from the amino acid sequence shown in SEQ ID NO:2 or a biologically active variant thereof, as defined above, or at least 6, 10, 15, 20, 25, 50, 75, 100, 125, 150, 175, 200, 225, 250, 275, 300, 325, 350, 375, 400, 425, 450, 475, 500, or 502 contiguous amino acids selected from the amino acid sequence shown in SEQ ID NO:4 or a biologically active variant thereof, as defined above. The first polypeptide segment also can comprise full-length prolyl 4-hydroxylase protein.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the prolyl 4-hydroxylase polypeptide-encoding sequence and the heterologous protein sequence, so that the prolyl 4-hydroxylase polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from SEQ ID NO:1 or SEQ ID NO:3 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human prolyl 4-hydroxylase polypeptides can be obtained using prolyl 4-hydroxylase polypeptide polynucleotides (described below) to make suitable probes or primers for screening cDNA expression libraries from other species, such as mice, monkeys, or yeast, identifying cDNAs which encode homologs of prolyl 4-hydroxylase polypeptides, and expressing the cDNAs as is known in the art.

Polynucleotides

A prolyl 4-hydroxylase polynucleotide can be single- or double-stranded and comprises a coding sequence or the complement of a coding sequence for a prolyl 4-hydroxylase polypeptide having either an amino acid sequence shown in SEQ ID NOS:2 or 4 or a biologically active variant thereof. A coding sequence for SEQ ID NO:2 is shown in SEQ ID NO:1; a coding sequence for SEQ ID NO:4 is shown in SEQ ID NO:3.

Degenerate nucleotide sequences encoding human prolyl 4-hydroxylase polypeptides, as well as homologous nucleotide sequences which are at least about 50, 55, 60, 65, 70, preferably about 75, 90, 96, 98, or 99% identical to the nucleotide sequences shown in SEQ ID NO:1 and SEQ ID NO:3 or their complements also are prolyl 4-hydroxylase polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of prolyl 4-hydroxylase polynucleotides that encode biologically active prolyl 4-hydroxylase polypeptides also are prolyl 4-hydroxylase polynucleotides. Polynucleotide fragments comprising at least 8, 9, 10, 11, 12, 15, 20, or 25 contiguous nucleotides of SEQ ID NO: 1 or SEQ ID NO:3 or their complements also are prolyl 4-hydroxylase polynucleotides. These fragments can be used, for example, as hybridization probes or as antisense oligonucleotides.

Identification of Polynucleotide Variants and Homologs

Variants and homologs of the prolyl 4-hydroxylase polynucleotides described above also are prolyl 4-hydroxylase polynucleotides. Typically, homologous prolyl 4-hydroxylase polynucleotide sequences can be identified by hybridization of candidate polynucleotides to known prolyl 4-hydroxylase polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the prolyl 4-hydroxylase polynucleotides disclosed herein also can be identified by making suitable probes or primers and screening cDNA expression libraries from other species, such as mice, monkeys, or yeast. Human variants of prolyl 4-hydroxylase polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123, 1973). Variants of human prolyl 4-hydroxylase polynucleotides or prolyl 4-hydroxylase polynucleotides of other species can therefore be identified by hybridizing a putative homologous prolyl 4-hydroxylase polynucleotide with a polynucleotide having a nucleotide sequence of SEQ ID NO:1 or SEQ ID NO:3 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to prolyl 4-hydroxylase polynucleotides or their complements following stringent hybridization and/or wash conditions also are prolyl 4-hydroxylase polynucleotides. Stringent wash conditions are well known and understood in the art and are disclosed, for example, in Sambrook et al., Molecular Cloning: A Laboratory Manual, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a prolyl 4-hydroxylase polynucleotide having a nucleotide sequence shown in SEQ ID NO:1 or SEQ ID NO:3 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):
Tₘ=81.5° C.−16.6(log10[Na⁺])+0.41(% G+C)−0.63(% formamide)−600/1),

where 1=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

A prolyl 4-hydroxylase polynucleotide can be isolated free of other cellular components such as membrane components, proteins, and lipids. Polynucleotides can be made by a cell and isolated using standard nucleic acid purification techniques or can be synthesized using an amplification technique (such as the polymerase chain reaction) or using an automatic synthesizer.

Methods for isolating polynucleotides are routine and are known in the art. Any such technique for obtaining a polynucleotide can be used to obtain isolated prolyl 4-hydroxylase polynucleotides. For example, restriction enzymes and probes can be used to isolate polynucleotide fragments, which comprise prolyl 4-hydroxylase nucleotide sequences. Isolated polynucleotides are in preparations that are free or at least 70, 80, or 90% free of other molecules.

Human prolyl 4-hydroxylase cDNA molecules can be made with standard molecular biology techniques, using prolyl 4-hydroxylase mRNA as a template. Human prolyl 4-hydroxylase cDNA molecules can thereafter be replicated using molecular biology techniques known in the art and disclosed in manuals such as Sambrook et al. (1989). An amplification technique, such as PCR, can be used to obtain additional copies of prolyl 4-hydroxylase polynucleotides, using either human genomic DNA or cDNA as a template.

Alternatively, synthetic chemistry techniques can be used to synthesize prolyl 4-hydroxylase polynucleotides. The degeneracy of the genetic code allows alternate nucleotide sequences to be synthesized which will encode a prolyl 4-hydroxylase polypeptide having an amino acid sequence shown in SEQ ID NO:2 or SEQ ID NO:4 or a biologically active variant thereof.

Extending Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus. Sarkar, PCR Methods Applic. 2, 318-322, 1993; Triglia et al., Nucleic Acids Res. 16, 8186, 1988; Lagerstrom et al., PCR Methods Applic. 1, 111-119, 1991; Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). See WO 01/98340.

Obtaining Polynucleotides

Human prolyl 4-hydroxylase polypeptides can be obtained, for example, by purification from human cells, by expression of prolyl 4-hydroxylase polynucleotides, or by direct chemical synthesis.

Protein Purification

Human prolyl 4-hydroxylase polypeptides can be purified from any human cell which expresses the receptor, including host cells which have been transfected with prolyl 4-hydroxylase polynucleotides. A purified prolyl 4-hydroxylase polypeptide is separated from other compounds that normally associate with the prolyl 4-hydroxylase polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

A preparation of purified prolyl 4-hydroxylase polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a human prolyl 4-hydroxylase polynucleotide, the polynucleotide can be inserted into an expression vector which contains the necessary elements for the transcription and translation of the inserted coding sequence. Methods which are well known to those skilled in the art can be used to construct expression vectors containing sequences encoding prolyl 4-hydroxylase polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., Current Protocols In Molecular Biology, John Wiley & Sons, New York, N.Y., 1989.

A variety of expression vector/host systems can be utilized to contain and express sequences encoding a human prolyl 4-hydroxylase polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems. See WO 01/98340.

Host Cells

A host cell strain can be chosen for its ability to modulate the expression of the inserted sequences or to process the expressed prolyl 4-hydroxylase polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells that have specific cellular machinery and characteristic mechanisms for post-translational activities (e.g., CHO, HeLa, MDCK, HEK293, and W138) are available from the American Type Culture Collection (ATCC; 10801 University Boulevard, Manassas, Va. 20110-2209) and can be chosen to ensure the correct modification and processing of the foreign protein. See WO 01/98340.

Alternatively, host cells which contain a human prolyl 4-hydroxylase polynucleotide and which express a human prolyl 4-hydroxylase polypeptide can be identified by a variety of procedures known to those of skill in the art. Examples include enzyme-linked immunosorbent assay (ELISA), radioimmunoassay (RIA), and fluorescence activated cell sorting (FACS). Hampton et al., Serological Methods: A Laboratory Manual, APS Press, St. Paul, Minn., 1990); Maddox et al., J. Exp. Med. 158, 1211-1216, 1983). See WO 01/98340.

A wide variety of labels and conjugation techniques are known by those skilled in the art and can be used in various nucleic acid and amino acid assays. Means for producing labeled hybridization or PCR probes for detecting sequences related to polynucleotides encoding prolyl 4-hydroxylase polypeptides include oligolabeling, nick translation, end-labeling, or PCR amplification using a labeled nucleotide. Alternatively, sequences encoding a human prolyl 4-hydroxylase polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding a human prolyl 4-hydroxylase polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode prolyl 4-hydroxylase polypeptides can be designed to contain signal sequences which direct secretion of soluble prolyl 4-hydroxylase polypeptides through a prokaryotic or eukaryotic cell membrane or which direct the membrane insertion of membrane-bound prolyl 4-hydroxylase polypeptide. See WO 01/98340.

Chemical Synthesis

Sequences encoding a human prolyl 4-hydroxylase polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, a human prolyl 4-hydroxylase polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or automation. Automated synthesis can be achieved, for example, using Applied Biosystems 431A Peptide Synthesizer (Perkin Elmer). Optionally, fragments of prolyl 4-hydroxylase polypeptides can be separately synthesized and combined using chemical methods to produce a full-length molecule. See WO 01/98340.

As will be understood by those of skill in the art, it may be advantageous to produce prolyl 4-hydroxylase polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter prolyl 4-hydroxylase polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a human prolyl 4-hydroxylase polypeptide. “Antibody” as used herein includes intact immunoglobulin molecules, as well as fragments thereof, such as Fab, F(ab′)₂, and Fv, which are capable of binding an epitope of a human prolyl 4-hydroxylase polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a human prolyl 4-hydroxylase polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody that specifically binds to the immunogen.

Typically, an antibody that specifically binds to a human prolyl 4-hydroxylase polypeptide provides a detection signal at least 5-, 10-, or 20-fold higher than a detection signal provided with other proteins when used in an immunochemical assay. Preferably, antibodies that specifically bind to prolyl 4-hydroxylase polypeptides do not detect other proteins in immunochemical assays and can immunoprecipitate a human prolyl 4-hydroxylase polypeptide from solution. See WO 01/98340.

Antisense Oligonucleotides

Antisense oligonucleotides are nucleotide sequences that are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of prolyl 4-hydroxylase gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of prolyl 4-hydroxylase gene expression can be obtained by designing antisense oligonucleotides that will form duplexes to the control, 5″, or regulatory regions of the prolyl 4-hydroxylase gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, Molecular And Immunologic Approaches, Futura Publishing Co., Mt. Kisco, N.Y., 1994). An antisense oligonucleotide also can be designed to block translation of mRNA by preventing the transcript from binding to ribosomes. See WO 01/98340.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a human prolyl 4-hydroxylase polynucleotide can be used to generate ribozymes that will specifically bind to mRNA transcribed from a prolyl 4-hydroxylase polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). See WO 01/98340.

Differentially Expressed Genes

Described herein are methods for the identification of genes whose products interact with a human prolyl 4-hydroxylase. Such genes may be genes that are differentially expressed in disorders including, but not limited to, cardiovascular disorders, cancer, inflammatory diseases, fibrotic disorders, anima, and CNS disorders, such as stroke. Such genes may be genes that are differentially regulated in response to manipulations relevant to the progression or treatment of such diseases. Additionally, such genes may have a temporally modulated expression (increased or decreased) at different stages of tissue or organism development. A differentially expressed gene may also have its expression modulated under control versus experimental conditions. In addition, the human prolyl 4-hydroxylase genes or gene products may themselves be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

To identify differentially expressed genes, total RNA or preferably mRNA, is isolated from tissues of interest. For example, RNA samples are obtained from tissues of experimental subjects and from corresponding tissues of control subjects. Any RNA isolation technique that does not select against the isolation of mRNA may be utilized for the purification of such RNA samples. See, for example, Ausubel et al., eds., Current Protocols In Molecular Biology, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples that represent RNA produced by differentially expressed genes are identified by methods well known to those of skill in the art. They include, for example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad. Sci. U.S.A. 88, 2825, 1984), and, preferably, differential display (Liang & Pardee, Science 257, 967-71, 1992; U.S. Pat. No. 5,262,311).

The differential expression information may itself suggest relevant methods for the treatment of disorders involving a human prolyl 4-hydroxylase. For example, treatment may include modulation of expression of the differentially expressed genes and/or a gene encoding a human prolyl 4-hydroxylase. The differential expression information may indicate whether the expression or activity of the differentially expressed gene or gene product or the human prolyl 4-hydroxylase gene or gene product are up-regulated or down-regulated.

Screening Methods

The invention provides assays for screening test compounds that bind to or modulate the activity of a prolyl 4-hydroxylase polypeptide or a prolyl 4-hydroxylase polynucleotide. A test compound preferably binds to a prolyl 4-hydroxylase polypeptide or polynucleotide. More preferably, a test compound decreases or increases prolyl 4-hydroxylase activity by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, can be produced recombinantly, or can be synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is typically used with polypeptide libraries, while the other four approaches are typically used with polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al., Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-21, 1992), or on beads (Lam, Nature 354, 82-84, 1991), chips (Fodor, Nature 364, 555-56, 1993), bacteria or spores (Ladner, U.S. Pat. No. 5,223,409), plasmids (Cull et al., Proc. Natl. Acad. Sci. U.S.A. 89, 1865-69, 1992), or phage (Scott & Smith, Science 249, 38690, 1990; Devlin, Science 249, 40406, 1990); Cwirla et al., Proc. Natl. Acad. Sci. 97, 6378-82, 1990; Felici, J. Mol. Biol. 222, 301-10, 1991; and Ladner, U.S. Pat. No. 5,223,409).

High Throughput Screening

Test compounds can be screened using high throughput screening for the ability to bind to prolyl 4-hydroxylase polypeptides or polynucleotides or to affect prolyl 4-hydroxylase activity or prolyl 4-hydroxylase gene expression. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 ml. In addition to the plates, many instruments, materials, pipettors, robotics, plate washers, and plate readers are commercially available to fit the 96-well format.

Alternatively, “free format assays,” i.e., assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another method is taught by Salmon et al., Molecular Diversity 2, 57-63 (1996). In this method, combinatorial libraries are screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule that binds to and occupies, for example, the active site of a prolyl 4-hydroxylase polypeptide, such that normal enzymatic activity is prevented. Examples of such small molecules include, but are not limited to, small peptides or peptide-like molecules.

In binding assays, either the test compound or the prolyl 4-hydroxylase polypeptide can comprise a detectable label, such as a fluorescent, radioisotopic, chemiluminescent, or enzymatic label, such as horseradish peroxidase, alkaline phosphatase, or luciferase. A test compound that is bound to the prolyl 4-hydroxylase polypeptide can then be detected, for example, by direct counting of radioemmission, by scintillation counting, or by determining conversion of an appropriate substrate to a detectable product.

Alternatively, binding of a test compound to a prolyl 4-hydroxylase polypeptide can be determined without labeling either of the interactants. For example, a microphysiometer can be used to detect binding of a test compound with a prolyl 4-hydroxylase polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a prolyl 4-hydroxylase polypeptide (McConnell et al., Science 257, 1906-12, 1992).

The ability of a test compound to bind to a prolyl 4-hydroxylase polypeptide also can be determined using a technology such as real-time Bimolecular Interaction Analysis (BIA) (Sjolander & Urbaniczky, Anal. Chem. 63, 2338-45, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a prolyl 4-hydroxylase polypeptide can be used as a “bait protein” in a two-hybrid assay or three-hybrid assay (see, e.g., U.S. Pat. No. 5,283,317; Zervos et al., Cell 72, 223-32, 1993; Madura et al., J. Biol. Chem. 268, 12046-54, 1993; Bartel et al., BioTechniques 14, 92024, 1993; Iwabuchi et al., Oncogene 8, 1693-96, 1993; and Brent W094/10300), to identify other proteins which bind to or interact with the prolyl 4-hydroxylase polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a prolyl 4-hydroxylase polypeptide is fused to a polynucleotide encoding the DNA binding domain of a known transcription factor (e.g., GAL-4). In the other construct a DNA sequence that encodes an unidentified protein (“prey” or “sample”) is fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein that interacts with the prolyl 4-hydroxylase polypeptide.

It may be desirable to immobilize either the prolyl 4-hydroxylase polypeptide (or polynucleotide) or the test compound to facilitate separation of bound from unbound forms of one or both of the interactants, as well as to accommodate automation of the assay. Thus, either a prolyl 4-hydroxylase polypeptide (or polynucleotide) or a test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the polypeptide, polynucleotide, or test compound to the solid support, including the use of covalent and non-covalent linkages, passive absorption, or pairs of binding moieties attached respectively to the polypeptide, polynucleotide, or test compound and the solid support. Test compounds are preferably bound to the solid support in an array, so that the location of individual test compounds can be tracked. Binding of a test compound to a prolyl 4-hydroxylase polypeptide or polynucleotide can be accomplished in any vessel suitable for containing the reactants. Examples of such vessels include microtiter plates, test tubes, and microcentrifuge tubes.

In one embodiment, the prolyl 4-hydroxylase polypeptide is a fusion protein comprising a domain that allows the prolyl 4-hydroxylase polypeptide to be bound to a solid support. For example, glutathione-S-transferase fusion proteins can be adsorbed onto glutathione sepharose beads (Sigma Chemical, St. Louis, Mo.) or glutathione-derivatized microtiter plates, which are then combined with the test compound or the test compound and the non-adsorbed prolyl 4-hydroxylase polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, a prolyl 4-hydroxylase polypeptide, or polynucleotide, or a test compound can be immobilized utilizing conjugation of biotin and streptavidin. Biotinylated prolyl 4-hydroxylase polypeptides, polynucleotides, or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96-well plates (Pierce Chemical). Alternatively, antibodies which specifically bind to a prolyl 4-hydroxylase polypeptide, polynucleotide, or a test compound, but which do not interfere with a desired binding site, such as the active site of the prolyl 4-hydroxylase polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the prolyl 4-hydroxylase polypeptide or test compound, enzyme-linked assays which rely on detecting an activity of the prolyl 4-hydroxylase polypeptide, and SDS gel electrophoresis under non-reducing conditions.

Screening for test compounds which bind to a prolyl 4-hydroxylase polypeptide or polynucleotide also can be carried out in an intact cell. Any cell which comprises a prolyl 4-hydroxylase polypeptide or polynucleotide can be used in a cell-based assay system. A prolyl 4-hydroxylase polynucleotide can be naturally occurring in the cell or can be introduced using techniques such as those described above. Binding of the test compound to a prolyl 4-hydroxylase polypeptide or polynucleotide is determined as described above.

Enzyme Assays

Test compounds can be tested for the ability to increase or decrease the enzymatic activity of a human prolyl 4-hydroxylase polypeptide. Prolyl 4-hydroxylase activity can be measured, for example, as described in Kivirikko & Myllylä, Methods Enzymol. 82, 245-304, 1982, or Cuncliffe et al., Biochem. J. 240, 617-19, 1986.

Enzyme assays can be carried out, for example, after contacting a purified prolyl 4-hydroxylase polypeptide with a test compound. A test compound that decreases a prolyl 4-hydroxylase activity of a prolyl 4-hydroxylase polypeptide by at least about 10, preferably about 50, more preferably about 75, 90, or 100% is identified as a potential therapeutic agent for decreasing prolyl 4-hydroxylase activity. A test compound which increases a prolyl 4-hydroxylase activity of a human prolyl 4-hydroxylase polypeptide by at least about 10, preferably about 50, more preferably about 75, 90, or 100% is identified as a potential therapeutic agent for increasing human prolyl 4-hydroxylase activity.

Gene Expression

In another embodiment, test compounds that increase or decrease prolyl 4-hydroxylase gene expression are identified. A prolyl 4-hydroxylase polynucleotide is contacted with a test compound, and the expression of an RNA or polypeptide product of the prolyl 4-hydroxylase polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of prolyl 4-hydroxylase mRNA or polypeptide expression in the cells can be determined by methods well known in the art for detecting mRNA or polypeptides. Either qualitative or quantitative methods can be used. The presence of polypeptide products of a prolyl 4-hydroxylase polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a prolyl 4-hydroxylase polypeptide.

Such screening can be carried out either in a cell-free assay system or in an intact cell. Any cell that expresses a prolyl 4-hydroxylase polynucleotide can be used in a cell-based assay system. The prolyl 4-hydroxylase polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions that can be administered to a patient to achieve a therapeutic effect. Pharmaceutical compositions of the invention can comprise, for example, a prolyl 4-hydroxylase polypeptide, prolyl 4-hydroxylase polynucleotide, ribozymes or antisense oligonucleotides, antibodies which specifically bind to a prolyl 4-hydroxylase polypeptide, or mimetics, activators, or inhibitors of a prolyl 4-hydroxylase polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries that facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of Remington's Pharmaceutical Sciences (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

The novel human prolyl 4-hydroxylases PH-1 and PH-2 can be regulated to treat cardiovascular disorders, anemia, cancer, CNS disorders, inflammatory diseases, and fibrotic diseases. PH-1 and PH-2 show a widespread tissue distribution which, together with their putative functions, suggests a central role in oxygen sensing and/or posttranslational modification of collagen. As prolyl 4-hydroxylases specific for hypoxia inducible transcription factors (HIFs), PH-1 and PH-2 may play a central role in the regulation of those genes that are transcriptionally regulated in response to changes of tissue oxygenation. Among those HIF-regulated genes, for example, vascular endothelial growth factor (VEGF) is of central importance for the de novo formation of blood vessels (angiogenesis), and erythropoietin (Epo) is the key regulator of the formation of red blood cells (erythropoiesis) in the bone marrow. As prolyl 4-hydroxylases that catalyze the hydroxylation of proline residues in pro-collagen, PH-1 and PH-2 are of crucial importance for the correct folding of the newly formed collagen molecules and therefore may be involved in diseases which are characterized by an increased deposition of collagen into the diseased tissue.

Cancer

Cancer is a disease fundamentally caused by oncogenic cellular transformation. There are several hallmarks of transformed cells that distinguish them from their normal counterparts and underlie the pathophysiology of cancer. These include uncontrolled cellular proliferation, unresponsiveness to normal death-inducing signals (immortalization), increased cellular motility and invasiveness, increased ability to recruit blood supply through induction of new blood vessel formation (angiogenesis), genetic instability, and dysregulated gene expression. Various combinations of these aberrant physiologies, along with the acquisition of drug-resistance frequently lead to an intractable disease state in which organ failure and patient death ultimately ensue.

Most standard cancer therapies target cellular proliferation and rely on the differential proliferative capacities between transformed and normal cells for their efficacy. This approach is hindered by the facts that several important normal cell types are also highly proliferative and that cancer cells frequently become resistant to these agents. Thus, the therapeutic indices for traditional anti-cancer therapies rarely exceed 2.0.

The advent of genomics-driven molecular target identification has opened up the possibility of identifying new cancer-specific targets for therapeutic intervention that will provide safer, more effective treatments for cancer patients. Thus, newly discovered tumor-associated genes and their products can be tested for their role(s) in disease and used as tools to discover and develop innovative therapies. Genes playing important roles in any of the physiological processes outlined above can be characterized as cancer targets.

Genes or gene fragments identified through genomics can readily be expressed in one or more heterologous expression systems to produce functional recombinant proteins. These proteins are characterized in vitro for their biochemical properties and then used as tools in high-throughput molecular screening programs to identify chemical modulators of their biochemical activities. Activators and/or inhibitors of target protein activity can be identified in this manner and subsequently tested in cellular and in vivo disease models for anti-cancer activity. Optimization of lead compounds with iterative testing in biological models and detailed pharmacokinetic and toxicological analyses form the basis for drug development and subsequent testing in humans.

Cardiovascular Disorders

Cardiovascular diseases include the following disorders of the heart and the vascular system: congestive heart failure, myocardial infarction, ischemic diseases of the heart, all kinds of atrial and ventricular arrhythmias, hypertensive vascular diseases, and peripheral vascular diseases.

Heart failure is defined as a pathophysiologic state in which an abnormality of cardiac function is responsible for the failure of the heart to pump blood at a rate commensurate with the requirement of the metabolizing tissue. It includes all forms of pumping failure, such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included, as well as the acute treatment of MI and the prevention of complications.

Ischemic diseases are conditions in which the coronary flow is restricted resulting in a perfusion which is inadequate to meet the myocardial requirement for oxygen. This group of diseases includes stable angina, unstable angina, and asymptomatic ischemia.

Arrhythmias include all forms of atrial and ventricular tachyarrhythmias (atrial tachycardia, atrial flutter, atrial fibrillation, atrio-ventricular reentrant tachycardia, preexcitation syndrome, ventricular tachycardia, ventricular flutter, and ventricular fibrillation), as well as bradycardic forms of arrhythmias.

Vascular diseases include primary as well as all kinds of secondary arterial hypertension (renal, endocrine, neurogenic, others). The disclosed genes and their products may be used as drug targets for the treatment of hypertension as well as for the prevention of all complications. Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon, and venous disorders.

Inflammatory Diseases

Inflammatory diseases are characterized by tissue alteration and/or destruction by cells and/or products of the body's immune defense system, either in response to exogenous agents, such as viral or bacterial pathogens or chemical agents, and/or in response to normal or altered structures (e.g., auto-immune diseases). Inflammatory tissue alterations include delivery of plasma water as consequence of disturbed blood vessel permeability, deposition of immune defense cells, deposition of collagen with tissue induration and scar formation, destruction of tissue, and de novo formation of blood vessels. Inflammatory diseases include acute and chronic alterations of the joints, such as rheumatoid arthritis, of the skin, such as psoriasis of the heart and other inner organs, such as lupus erythematosus, and forms of myocarditis. The disclosed genes and their products may be used as drug targets for the treatment of inflammatory diseases.

Fibrotic Disorders

Fibrotic disorders originate either as a secondary response to tissue alterations, such as toxic or inflammatory destruction of the liver, or as primary lesions without discernible etiology. They are characterized by overproduction and deposit of collagen into the interstitium of the diseased organs, resulting in severely impaired organ function. Fibrotic disorders include fibrotic alterations of skin, liver, lung, and heart.

Anemia

Anemias are characterized by a lack of oxygen-transporting red blood cells. This leads to an impaired tissue oxygen supply. The lack of red blood cells can be the result of bleeding, of increased destruction of red blood cells (e.g., due to toxic agents), of decreased red blood cell stability, or to decreased de novo formation of red blood cells in the bone marrow. Impaired de novo formation can be the result of exposure to toxic agents (e.g., cancer chemotherapeutic agents), infiltration of the bone marrow by cancer cells, or a lack of erythropoietin, which is an indispensable growth factor for red blood cell formation. Erythropoietin is mainly, but not exclusively, secreted from the kidney. Therefore, the latter kind of anemia is mainly, but not exclusively, observed in patients with alterations of the kidneys.

CNS Disorders

Central and peripheral nervous system disorders also can be treated, such as primary and secondary disorders after brain injury or stroke. The disclosed genes and their products can be used as drug targets for the prevention and the treatment of all CNS disorders that are due to alterations of brain blood vessels and/or due to ischemic and/or hypoxic alterations of the CNS.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a prolyl 4-hydroxylase polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects prolyl 4-hydroxylase activity can be administered to a human cell, either in vitro or in vivo, to reduce prolyl 4-hydroxylase activity. The reagent preferably binds to an expression product of a human prolyl 4-hydroxylase gene. If the expression product is a protein, the reagent is preferably an antibody. For treatment of human cells ex vivo, an antibody can be added to a preparation of stem cells that have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 mg of DNA per 16 nmole of liposome delivered to about 10⁶ cells, more preferably about 1.0 mg of DNA per 16 nmole of liposome delivered to about 10⁶ cells, and even more preferably about 2.0 mg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods that are standard in the art (see, for example, U.S. Pat. No. 5,705,151). Preferably, from about 0.1 mg to about 10 mg of polynucleotide is combined with about 8 nmol of liposomes, more preferably from about 0.5 mg to about 5 mg of polynucleotides are combined with about 8 nmol liposomes, and even more preferably about 1.0 mg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al., Trends in Biotechnol 11, 202-05, 1993; Chiou et al., Gene Therapeutics: Methods and Applications of Direct Gene Transfer, J. A. Wolff, ed., 1994; Wu & Wu, J. Biol. Chem. 263, 621-24, 1988; Wu et al., J. Biol. Chem. 269, 542-46, 1994; Zenke et al., Proc. Natl. Acad. Sci. U.S.A. 87, 3655-59, 1990; Wu et al., J. Biol. Chem. 266, 338-42, 1991.

Determination of a Therapeutically Effective Dose

The determination of a therapeutically effective dose is well within the capability of those skilled in the art. A therapeutically effective dose refers to that amount of active ingredient which increases or decreases prolyl 4-hydroxylase activity relative to the prolyl 4-hydroxylase activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors that can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 mg to about 50 mg/kg, about 50 mg to about 5 mg/kg, about 100 mg to about 500 mg/kg of patient body weight, and about 200 to about 250 mg/kg of patient body weight. For administration of polynucleotides encoding single-chain antibodies, effective in vivo dosages are in the range of about 100 ng to about 200 ng, 500 ng to about 50 mg, about 1 mg to about 2 mg, about 5 mg to about 500 mg, and about 20 mg to about 100 mg of DNA.

If the expression product is mRNA, the reagent is preferably an antisense oligonucleotide or a ribozyme. Polynucleotides that express antisense oligonucleotides or ribozymes can be introduced into cells by a variety of methods, as described above.

Preferably, a reagent reduces expression of a prolyl 4-hydroxylase gene or the activity of a prolyl 4-hydroxylase polypeptide by at least about 10, preferably about 50, more preferably about 75, 90, or 100% relative to the absence of the reagent. The effectiveness of the mechanism chosen to decrease the level of expression of a prolyl 4-hydroxylase gene or the activity of a prolyl 4-hydroxylase polypeptide can be assessed using methods well known in the art, such as hybridization of nucleotide probes to prolyl 4-hydroxylase-specific mRNA, quantitative RT-PCR, immunologic detection of a prolyl 4-hydroxylase polypeptide, or measurement of prolyl 4-hydroxylase activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Human prolyl 4-hydroxylase also can be used in diagnostic assays for detecting diseases and abnormalities or susceptibility to diseases and abnormalities related to the presence of mutations in the nucleic acid sequences that encode the enzyme. For example, differences can be determined between the cDNA or genomic sequence encoding a prolyl 4-hydroxylase in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. U.S.A. 85, 4397-401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel electrophoresis and DNA sequencing, mutations can also be detected by in situ analysis.

Altered levels of prolyl 4-hydroxylase also can be detected in various tissues. Assays used to detect levels of the enzyme in a body sample, such as blood or a tissue biopsy, are well known to those of skill in the art and include radioimmunoassays, competitive binding assays, Western blot analysis, and ELISA assays.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

Expression of Recombinant Human Prolyl 4-hydroxylase

The Pichia pastoris expression vector pPICZB (Invitrogen, San Diego, Calif.) is used to produce large quantities of recombinant human prolyl 4-hydroxylase polypeptides in yeast. The prolyl 4-hydroxylase-encoding DNA sequence is derived from SEQ ID NO:1 or SEQ ID NO:3. Before insertion into vector pPICZB, the DNA sequence is modified by well known methods in such a way that it contains at its 5′-end an initiation codon and at its 3′-end an enterokinase cleavage site, a His6 reporter tag and a termination codon. Recognition sequences for restriction endonucleases are added at both termini. After digestion of the multiple cloning site of pPICZ B with the corresponding restriction enzymes, the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, Calif.) according to manufacturer's instructions. Purified human prolyl 4-hydroxylase polypeptide is obtained.

### EXAMPLE 2

Identification of Test Compounds that Bind to Prolyl 4-hydroxylase Polypeptides

Purified prolyl 4-hydroxylase polypeptides comprising a glutathione-S-transferase protein and absorbed onto glutathione-derivatized wells of 96-well microtiter plates are contacted with test compounds from a small molecule library at pH 7.0 in a physiological buffer solution. Human prolyl 4-hydroxylase polypeptides comprise the amino acid sequence shown in SEQ ID NO:2 or SEQ ID NO:4. The test compounds comprise a fluorescent tag. The samples are incubated for 5 minutes to one hour. Control samples are incubated in the absence of a test compound.

The buffer solution containing the test compounds is washed from the wells. Binding of a test compound to a prolyl 4-hydroxylase polypeptide is detected by fluorescence measurements of the contents of the wells. A test compound that increases the fluorescence in a well by at least 15% relative to fluorescence of a well in which a test compound is not incubated is identified as a compound which binds to a prolyl 4-hydroxylase polypeptide.

### EXAMPLE 3

Identification of a Test Compound which Decreases Prolyl 4-hydroxylase Gene Expression

A test compound is administered to a culture of human cells transfected with a prolyl 4-hydroxylase expression construct and incubated at 37° C. for 10 to 45 minutes. A culture of the same type of cells that have not been transfected is incubated for the same time without the test compound to provide a negative control.

RNA is isolated from the two cultures as described in Chirgwin et al., Biochem. 18, 5294-99, 1979). Northern blots are prepared using 20 to 30 mg total RNA and hybridized with a ³²P-labeled prolyl 4-hydroxylase-specific probe at 65° C. in Express-hyb (CLONTECH). The probe comprises at least 11 contiguous nucleotides selected from the complement of SEQ ID NO:1 or SEQ ID NO:3. A test compound that decreases the prolyl 4-hydroxylase-specific signal relative to the signal obtained in the absence of the test compound is identified as an inhibitor of prolyl 4-hydroxylase gene expression.

### EXAMPLE 4

Identification of a Test Compound which Decreases or Increases Prolyl 4-hydroxylase Activity

A test compound is administered to a mixture of purified prolyl 4-hydroxylase and an appropriate reaction buffer and incubated at 37° C. for 10 to 45 minutes. A mixture of the same type but without test compound is used as a control. The prolyl 4-hydroxylase activity is measured using a method of Kivirikko & Myllylä, Methods Enzymol. 82, 245-304, 1982, or Cuncliffe et al., Biochem. J. 240, 617-19, 1986.

A test compound which decreases the prolyl 4-hydroxylase activity of the prolyl 4-hydroxylase relative to the prolyl 4-hydroxylase activity in the absence of the test compound is identified as an inhibitor of prolyl 4-hydroxylase activity. A test compound which increases the prolyl 4-hydroxylase activity of the prolyl 4-hydroxylase relative to the prolyl 4-hydroxylase activity in the absence of the test compound is identified as an activator of prolyl 4-hydroxylase activity.

### EXAMPLE 5

Tissue-Specific Expression of PH-1 and PH-2

The qualitative expression pattern of PH-1 and PH-2 in various tissues was determined by real time quantitative polymerase chain reaction. (TaqMan-PCR, Heid et al., Genome Res. 6 (10)) on an ABI Prism 7700 sequence detection instrument (Applied Biosystems, Inc.). One microgram of commercially available total RNA from various human tissues (Fa. Clontech) was digested with DNase I and reverse transcribed into cDNA using Superscript-II RT-PCR kit (Gibco, Inc.). Two and one-half percent of the obtained cDNA pool were used for each polymerase chain reaction.

The sequences of forward and reverse primers as designed by Primer Express 1.5 Software (Applied Biosystems, Inc.) were 5′-AGCCTCCTGGAAGAAGGCC-3′ (SEQ ID NO:5) and 5′-GGTAACAACCTCTCCCTTGCC-3′ (SEQ ID NO:6) for the quantification of PH-1, the fluorogenic probe used was 5′-6FAM-TGTCAGCTTTGTCTGTGCCTCGCA-TAMRA-3′ (SEQ ID NO:7). For PH-2, the forward and an reverse primer sequences were 5′-GCAGACTAAAGGTCTGGCCAA-3′ (SEQ ID NO:8) and 5′-ATAGGAACTGCGCCGTATCG-3′ (SEQ ID NO:9) respectively. The sequence of the fluorogenic probe for the detection of PH-2 was 5′-6FAM-TCTTGCCCCACCCCGCCA-TAMRA-3′ (SEQ ID NO:10). During PCR amplification, 5′-nucleolytic activity of Taq polymerase cleaves the probe separating the 5′ reporter fluorescent dye 6FAM (6-carboxy-fluorescein) from the 3′ quencher dye TAMRA (6-carboxy-tetramethyl-rhodamine). Because the fluorescence emission will increase in direct proportion to the amount of the specific amplified product, the exponential growth phase of PCR product can be detected and used to determine the initial template concentration. The threshold cycle (Ct), which correlates inversely with the target mRNA level, was measured as the cycle number at which the reporter fluorescent emission increases 10 standard deviations above background level. The mRNA levels of PH-1 and PH-2 were corrected for beta-actin mRNA levels to exclude different starting amounts of total RNA and calculated as relative expression using comparative dCt-method (described in TaqMan user guide, Applied Biosystems, Inc.). The tissue with the lowest expression level of PH-1 and PH-2 respectively was set as one. Relative expression values are depicted in FIG. 3 and FIG. 4 for PH-1 and PH-2, respectively.

### EXAMPLE 6

Proliferation Inhibition Assay: Antisense Oligonucleotides Suppress the Growth of Cancer Cell Lines

The cell line used for testing is the human colon cancer cell line HCT116. Cells are cultured in RPMI-1640 with 10-15% fetal calf serum at a concentration of 10,000 cells per milliliter in a volume of 0.5 ml and kept at 37° C. in a 95% air/5% CO₂ atmosphere.

Phosphorothioate oligoribonucleotides are synthesized on an Applied Biosystems Model 380B DNA synthesizer using phosphoroamidite chemistry. A sequence of 24 bases complementary to the nucleotides at position 1 to 24 of SEQ ID NO:1 or SEQ ID NO:3 is used as the test oligonucleotide. As a control, another (random) sequence is used: 5′-TCA ACT GAC TAG ATG TAC ATG GAC-3′ (SEQ ID NO:11). Following assembly and deprotection, oligonucleotides are ethanol-precipitated twice, dried, and suspended in phosphate buffered saline at the desired concentration. Purity of the oligonucleotides is tested by capillary gel electrophoresis and ion exchange HPLC. The purified oligonucleotides are added to the culture medium at a concentration of 10 μM once per day for seven days.

The addition of the test oligonucleotide for seven days results in significantly reduced expression of human prolyl 4-hydroxylase as determined by Western blotting. This effect is not observed with the control oligonucleotide. After 3 to 7 days, the number of cells in the cultures is counted using an automatic cell counter. The number of cells in cultures treated with the test oligonucleotide (expressed as 100%) is compared with the number of cells in cultures treated with the control oligonucleotide. The number of cells in cultures treated with the test oligonucleotide is not more than 30% of control, indicating that the inhibition of human prolyl 4-hydroxylase has an anti-proliferative effect on cancer cells.

EXAMPLE 7

In vivo Testing of Compounds/Target Validation

Acute Mechanistic Assays

Reduction in Mitogenic Plasma Hormone Levels

This non-tumor assay measures the ability of a compound to reduce either the endogenous level of a circulating hormone or the level of hormone produced in response to a biologic stimulus. Rodents are administered test compound (p.o., i.p., i.v., i.m., or s.c.). At a predetermined time after administration of test compound, blood plasma is collected. Plasma is assayed for levels of the hormone of interest. If the normal circulating levels of the hormone are too low and/or variable to provide consistent results, the level of the hormone may be elevated by a pre-treatment with a biologic stimulus (e.g., LHRH may be injected i.m. into mice at a dosage of 30 ng/mouse to induce a burst of testosterone synthesis). The timing of plasma collection would be adjusted to coincide with the peak of the induced hormone response. Compound effects are compared to a vehicle-treated control group. An F-test is preformed to determine if the variance is equal or unequal followed by a Student's t-test. Significance is p value ≦0.05 compared to the vehicle control group.

Hollow Fiber Mechanism of Action Assay

Hollow fibers are prepared with desired cell line(s) and implanted intraperitoneally and/or subcutaneously in rodents. Compounds are administered p.o., i.p., i.v., i.m., or s.c. Fibers are harvested in accordance with specific readout assay protocol, these may include assays for gene expression (bDNA, PCR, or Taqman), or a specific biochemical activity (e.g., cAMP levels). Results are analyzed by Student's t-test or Rank Sum test after the variance between groups is compared by an F-test, with significance at p≦0.05 as compared to the vehicle control group.

Subacute Functional In Vivo Assays

Reduction in Mass of Hormone Dependent Tissues

This is another non-tumor assay that measures the ability of a compound to reduce the mass of a hormone dependent tissue (i.e., seminal vesicles in males and uteri in females). Rodents are administered test compound (p.o., i.p., i.v., i.m., or s.c.) according to a predetermined schedule and for a predetermined duration (e.g., 1 week). At the termination of the study, animals are weighed, the target organ is excised, any fluid is expressed, and the weight of the organ is recorded. Blood plasma may also be collected. Plasma may be assayed for levels of a hormone of interest or for levels of test agent. Organ weights may be directly compared or they may be normalized for the body weight of the animal. Compound effects are compared to a vehicle-treated control group. An F-test is preformed to determine if the variance is equal or unequal followed by a Student's t-test. Significance is p value ≦0.05 compared to the vehicle control group.

Hollow Fiber Proliferation Assay

Hollow fibers are prepared with desired cell line(s) and implanted intraperitoneally and/or subcutaneously in rodents. Compounds are administered p.o., i.p., i.v., i.m., or s.c. Fibers are harvested in accordance with specific readout assay protocol. Cell proliferation is determined by measuring a marker of cell number (e.g., MTT or LDH). The cell number and change in cell number from the starting inoculum are analyzed by Student's t-test or Rank Sum test after the variance between groups is compared by an F-test, with significance at p≦0.05 as compared to the vehicle control group.

Anti-angiogenesis Models

Corneal Angiogenesis

Hydron pellets with or without growth factors or cells are implanted into a micropocket surgically created in the rodent cornea. Compound administration may be systemic or local (compound mixed with growth factors in the hydron pellet). Corneas are harvested at 7 days post implantation immediately following intracardiac infusion of colloidal carbon and are fixed in 10% formalin. Readout is qualitative scoring and/or image analysis. Qualitative scores are compared by Rank Sum test. Image analysis data is evaluated by measuring the area of neovascularization (in pixels) and group averages are compared by Student's t-test (2 tail). Significance is p≦0.05 as compared to the growth factor or cells only group.

Matrigel Angiogenesis

Matrigel, containing cells or growth factors, is injected subcutaneously. Compounds are administered p.o., i.p., i.v., i.m., or s.c. Matrigel plugs are harvested at predetermined time point(s) and prepared for readout. Readout is an ELISA-based assay for hemoglobin concentration and/or histological examination (e.g., vessel count, special staining for endothelial surface markers: CD31, factor-8). Readouts are analyzed by Student's t-test, after the variance between groups is compared by an F-test, with significance determined at p≦0.05 as compared to the vehicle control group.

Primary Antitumor Efficacy

Early Therapy Models

Subcutaneous Tumor

Tumor cells or fragments are implanted subcutaneously on Day 0. Vehicle and/or compounds are administered p.o., i.p., i.v., i.m., or s.c. according to a predetermined schedule starting at a time, usually on Day 1, prior to the ability to measure the tumor burden. Body weights and tumor measurements are recorded 2-3 times weekly. Mean net body and tumor weights are calculated for each data collection day. Anti-tumor efficacy may be initially determined by comparing the size of treated (T) and control (C) tumors on a given day by a Student's t-test, after the variance between groups is compared by an F-test, with significance determined at p≦0.05. The experiment may also be continued past the end of dosing in which case tumor measurements would continue to be recorded to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p≦0.05.

Intraperitoneal/Intracranial Tumor Models

Tumor cells are injected intraperitoneally or intracranially on Day 0. Compounds are administered p.o., i.p., i.v., i.m., or s.c. according to a predetermined schedule starting on Day 1. Observations of morbidity and/or mortality are recorded twice daily. Body weights are measured and recorded twice weekly. Morbidity/mortality data is expressed in terms of the median time of survival and the number of long-term survivors is indicated separately. Survival times are used to generate Kaplan-Meier curves. Significance is p≦0.05 by a log-rank test compared to the control group in the experiment.

Established Disease Model

Tumor cells or fragments are implanted subcutaneously and grown to the desired size for treatment to begin. Once at the predetermined size range, mice are randomized into treatment groups. Compounds are administered p.o., i.p., i.v., i.m., or s.c. according to a predetermined schedule. Tumor and body weights are measured and recorded 2-3 times weekly. Mean tumor weights of all groups over days post inoculation are graphed for comparison. An F-test is preformed to determine if the variance is equal or unequal followed by a Student's t-test to compare tumor sizes in the treated and control groups at the end of treatment. Significance is p≦0.05 as compared to the control group. Tumor measurements may be recorded after dosing has stopped to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p value≦0.05 compared to the vehicle control group.

Orthotopic Disease Models

Mammary Fat Pad Assay

Tumor cells or fragments, of mammary adenocarcinoma origin, are implanted directly into a surgically exposed and reflected mammary fat pad in rodents. The fat pad is placed back in its original position and the surgical site is closed. Hormones may also be administered to the rodents to support the growth of the tumors. Compounds are administered p.o., i.p., i.v., i.m., or s.c. according to a predetermined schedule. Tumor and body weights are measured and recorded 2-3 times weekly. Mean tumor weights of all groups over days post inoculation are graphed for comparison. An F-test is preformed to determine if the variance is equal or unequal followed by a Student's t-test to compare tumor sizes in the treated and control groups at the end of treatment. Significance is p≦0.05 as compared to the control group.

Tumor measurements may be recorded after dosing has stopped to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p value≦0.05 compared to the vehicle control group. In addition, this model provides an opportunity to increase the rate of spontaneous metastasis of this type of tumor. Metastasis can be assessed at termination of the study by counting the number of visible foci per target organ, or measuring the target organ weight. The means of these endpoints are compared by Student's t-test after conducting an F-test, with significance determined at p≦0.05 compared to the control group in the experiment.

Intraprostatic Assay

Tumor cells or fragments, of prostatic adenocarcinoma origin, are implanted directly into a surgically exposed dorsal lobe of the prostate in rodents. The prostate is externalized through an abdominal incision so that the tumor can be implanted specifically in the dorsal lobe while verifying that the implant does not enter the seminal vesicles. The successfully inoculated prostate is replaced in the abdomen and the incisions through the abdomen and skin are closed. Hormones may also be administered to the rodents to support the growth of the tumors. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (e.g., the lungs), or measuring the target organ weight (e.g., the regional lymph nodes). The means of these endpoints are compared by Student's t-test after conducting an F-test, with significance determined at p≦0.05 compared to the control group in the experiment.

Intrabronchial Assay

Tumor cells of pulmonary origin may be implanted intrabronchially by making an incision through the skin and exposing the trachea. The trachea is pierced with the beveled end of a 25 gauge needle and the tumor cells are inoculated into the main bronchus using a flat-ended 27 gauge needle with a 90° bend. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (e.g., the contralateral lung), or measuring the target organ weight. The means of these endpoints are compared by Student's t-test after conducting an F-test, with significance determined at p≦0.05 compared to the control group in the experiment.

Intracecal Assay

Tumor cells of gastrointestinal origin may be implanted intracecally by making an abdominal incision through the skin and externalizing the intestine. Tumor cells are inoculated into the cecal wall without penetrating the lumen of the intestine using a 27 or 30 gauge needle. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (e.g., the liver), or measuring the target organ weight. The means of these endpoints are compared by Student's t-test after conducting an F-test, with significance determined at p≦0.05 compared to the control group in the experiment.

Secondary (Metastatic) Antitumor Efficacy

Spontaneous Metastasis

Tumor cells are inoculated s.c. and the tumors allowed to grow to a predetermined range for spontaneous metastasis studies to the lung or liver. These primary tumors are then excised. Compounds are administered p.o., i.p., i.v., i.m., or s.c. according to a predetermined schedule which may include the period leading up to the excision of the primary tumor to evaluate therapies directed at inhibiting the early stages of tumor metastasis. Observations of morbidity and/or mortality are recorded daily. Body weights are measured and recorded twice weekly. Potential endpoints include survival time, numbers of visible foci per target organ, or target organ weight. When survival time is used as the endpoint the other values are not determined. Survival data is used to generate Kaplan-Meier curves. Significance is p≦0.05 by a log-rank test compared to the control group in the experiment. The mean number of visible tumor foci, as determined under a dissecting microscope, and the mean target organ weights are compared by Student's t-test after conducting an F-test, with significance determined at p≦0.05 compared to the control group in the experiment for both of these endpoints.

Forced Metastasis

Tumor cells are injected into the tail vein, portal vein, or the left ventricle of the heart in experimental (forced) lung, liver, and bone metastasis studies, respectively. Compounds are administered p.o., i.p., i.v., i.m., or s.c. according to a predetermined schedule. Observations of morbidity and/or mortality are recorded daily. Body weights are measured and recorded twice weekly. Potential endpoints include survival time, numbers of visible foci per target organ, or target organ weight. When survival time is used as the endpoint the other values are not determined. Survival data is used to generate Kaplan-Meier curves. Significance is p≦0.05 by a log-rank test compared to the control group in the experiment. The mean number of visible tumor foci, as determined under a dissecting microscope, and the mean target organ weights are compared by Student's t-test after conducting an F-test, with significance at p≦0.05 compared to the vehicle control group in the experiment for both endpoints.

### EXAMPLE 8

HIF Prolyl Hydroxylase Activity of PH-2

HIF-prolyl hydroxylase activity of PH-2 was examined in a cellular co-transfection assay in HEK/293 cells. In a typical experiment, cells were seeded at a density of 2×10⁴ cells per well in 96-well tissue culture plates (Greiner, Germany) and grown in DMEM F12 tissue culture medium (Gibco) supplemented with 10% fetal calf serum (PAA) and antibiotics for 24 hours at 37° C. in a humidified tissue culture incubator (Heraeus, Germany) in a 5% CO₂ atmosphere. Plasmid DNA was introduced into the HEK/293 cells by use of Lipofectamin reagent (Gibco) according to the manufacturer's instructions.

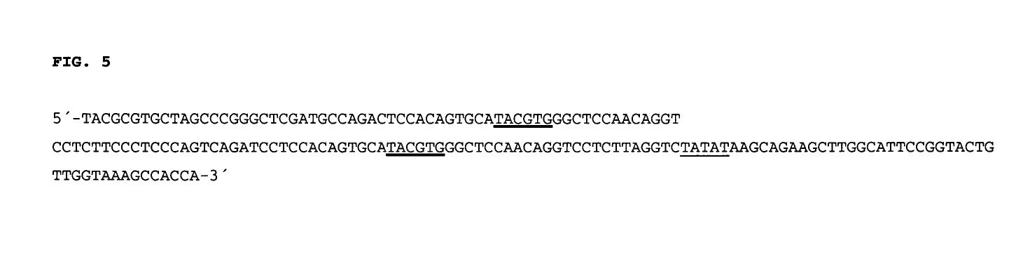

The following expression plasmids were used in transfection experiments. pCDNA3 cloning vectors were obtained from Invitrogen:
HIF-1alpha pcDNA3 containing the coding sequence of mouse hypoxia inducible factor-1 alpha (Gene bank accession number: NM_{—}010431).
HIF-1 alpha P402G pcDNA3 containing the coding sequence of mouse hypoxia inducible factor-1 alpha in which the HIF proline hydroxylase sensitive proline residue at position 402 was replaced with glycine by site-directed mutagenesis using QuickChange™ XL Site-Directed Mutagenesis Kit, Stratagene.
HIF-1 alpha P577G pcDNA3 containing the coding sequence of mouse hypoxia inducible factor-1alpha in which the HIF proline hydroxylase sensitive proline residue at position 577 (the ortholog of P564 of human HIF) was replaced with glycine by site-directed mutagenesis using QuickChange™ XL Site-Directed Mutagenesis Kit, Stratagene.
HIF-1 alpha P402/577G pcDNA3 containing the coding sequence of mouse hypoxia inducible factor-1alpha in which both HIF proline hydroxylase sensitive proline residues at positions 402 and 577 were replaced with glycine by site-directed mutagenesis using QuickChange™ XL Site-Directed Mutagenesis Kit, Stratagene.
HIF-2 alpha pcDNA3 containing the coding sequence of mouse hypoxia inducible factor-2 alpha (Gene bank accession number: AF045160).
HIF-RE2-luc, HIF reporter construct (constructed in pGL3, Promega) consisting of a minimal promoter containing a tandem of hypoxia responsible elements (FIG. 5, bold underlined) and the CMV promoter TATA box (FIG. 5, underlined) upstream of the firefly luciferase gene.
pRLTK (Promega) containing the coding sequence of renilla luciferase, used as transfection standard.
PH-alpha(I) pcDNA3 containing the coding sequence of human prolyl 4-hydroxylase alpha (I) (Gene bank accession number: U14620).
EGLN3 pcDNA3 containing the coding sequence of a recently identified human HIF-prolyl-hydroxylase (Epstein et al., Cell 107, 43-54, 2001; Bruick & McKnight, Science 294, 1337-40, 2001; gene bank accession number: XM_{—}052824).
PH-2 pcDNA3 containing the coding sequence from SEQ ID NO:3.

In a typical experiment, 30 ng of HIF-RE2 pGL3 luciferase reporter plasmid and 10 ng pRLTK internal standard were cotransfected with prolyl-4 hydroxylase and HIF pCDNA3 expression plasmids. The amount of transfected pCDNA3 plasmids was kept constant at 60 ng by filling up with pCDNA3 empty cloning vector. Twenty-four hours later, luciferase activity was measured in a lumibox equipped with a Hamamatsu camera (Hamamatsu Photonics, Japan) using after lysis of cells in luciferase buffer.

For Western blot analysis, cells were lysed using cell lysis buffer (10% glycerol, 5% 2-mercaptoethanol, 3.5% SDS, 62 mM Tris HCl, pH 6.8). Equal amounts of cell lysates were separated on 8% SDS polyacrylamide gels, and proteins were blotted onto nitrocellulose membranes (Optitran BA-S85, Schleicher & Schuell, Germany) at 10V for 30 min in a semidry blotting apparatus (BioRad). Detection of HIF-2 alpha protein was performed using a HIF-2 alpha specific rabbit antibody (Krieg et al., Oncogene 19, 5435-43, 2000). Binding of the HIF-2 alpha antibody was visualized by binding of a horseradish peroxidase conjugated anti rabbit antibody (Amersham) and subsequent enhanced chemiluminescence technique using ECLTM reagent (Amersham) according to the manufacturer's instructions.

Examination of PH-2 in Cotransfection Reporter Assays

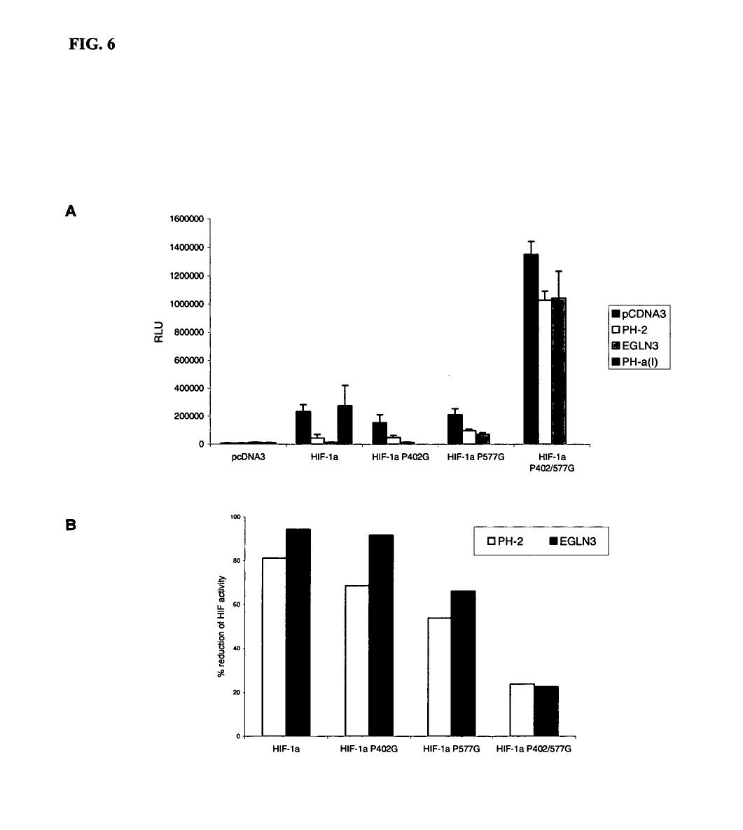

A luciferase reporter construct in which a tandem of hypoxia responsive elements linked to a minimal promoter drive firefly luciferase was cotransfected with HIF-1 alpha and HIF-2 alpha, respectively, and with the candidate HIF prolyl 4-hydroxylases EGLN3 and PH-2, respectively. Like EGLN3, PH-2 markedly reduced the transactivation activity of cotransfected HIF-1 alpha on the hypoxia responsive reporter gene construct by about 90% (EGLN3) and 80% (PH-2) (equal amounts of each plasmid were transfected) (FIGS. 6A, 6B). In contrast, the collagen prolyl 4-hydroxylase PH-alpha(I) did not reduce the activity of HIF-1 alpha in the same assay (FIG. 6A). An HIF-1 alpha mutant, in which glycine was substituted for P402, was equally active on the reporter construct and equally sensitive to EGLN3 and PH-2 as the wild type HIF-1 alpha. Mutation of P577 did not change the level of transactivation but rendered HIF-1 alpha less sensitive to EGLN3 and PH-2, respectively. The double mutant HIF-1 alpha, in contrast, was constitutively active, and this activity was almost completely resistant to cotransfected EGLN3 and PH-2, respectively (FIGS. 6A, 6B). These data demonstrate that PH-2, like EGLN3, reduces the activity of HIF-1alpha on a hypoxia responsive reporter via the critical proline residues of HIF. These findings suggest that in the cellular context PH-2 is active as HIF-proline 4-hydroxylase with a similar activity profile as EGLN3.

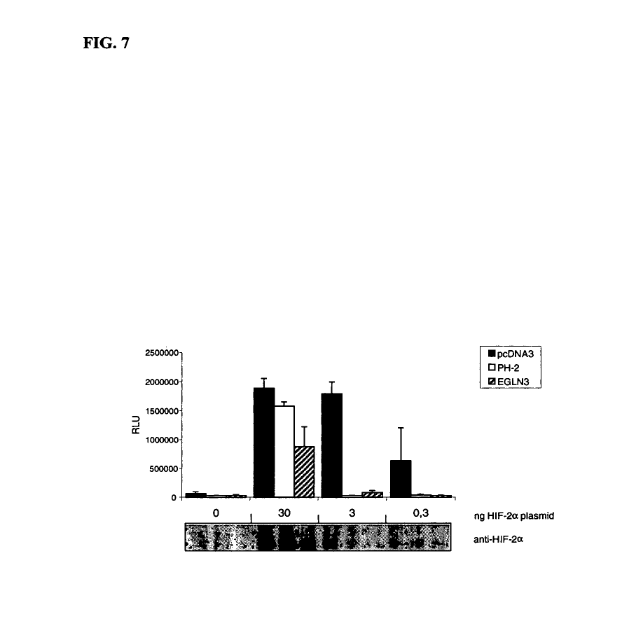

PH-2, like EGLN3, reduced the activity also of HIF-2 alpha on the hypoxia responsive reporter gene construct and was by a factor two less active than EGLN3 (equal amounts of each plasmid were transfected). Both proteins were less active on HIF-2 alpha than on HIF-1 alpha. However, when PH-2 and EGLN3, respectively, and HIF-2 alpha were cotransfected in a ratio 10:1, the activity of HIF-2 alpha was completely abolished (FIG. 7, upper panel). As demonstrated by Western blot analysis using an HIF-2 alpha specific polyclonal antibody, loss of HIF-2 alpha activity correlated with the disappearance of HIF-2 alpha protein in the presence of PH-2 and EGLN3, respectively (FIG. 7, lower panel).

Collectively, the data from cotransfection experiments indicate that PH-2 is a novel HIF prolyl hydroxylase that is involved in the degradation of HIFs under normoxia.

### REFERENCES

Targeting of HIF-alpha to the von Hippel-Lindau Ubiquitylation Complex by O₂-Regulated Prolyl Hydroxylation, Jaakkola P, et al, Science 2001 Apr. 20;292(5516):468-472.
The von Hippel-Lindau tumor suppressor protein, Ivan M, Kaelin W G Jr, Curr Opin Genet Dev 2001 February; 11(1):27-34
SIGNAL TRANSDUCTION: How Do Cells Sense Oxygen? Zhu H, Bunn H F, Science 2001 Apr. 20;292(5516):449-451
Cloning of the alpha subunit of prolyl 4-hydroxylase from Drosophila and expression and characterization of the corresponding enzyme tetramer with some unique properties. Annunen P et al, J Biol Chem 1999 Mar. 5;274(10):6790-6

1-55. (canceled)

56. A method of screening for candidate therapeutic agents that may be useful for treating cancer, a cardiovascular disorder, an inflammatory disorder, or a fibrotic disease comprising the steps of:
contacting a human prolyl 4-hydroxylase protein comprising the amino acid sequence shown in SEQ ID NO:2 or SEQ ID NO:4 with a test compound;
assaying for binding between the prolyl 4-hydroxylase protein and the test compound; and
identifying a test compound that binds to the prolyl 4-hydroxylase protein as a candidate therapeutic agent that may be useful for treating cancer, a cardiovascular disorder, an inflammatory disorder, or a fibrotic disease.

57. The method of claim 56 wherein either the test compound or the prolyl 4-hydroxylase protein comprises a detectable label.

58. The method of claim 56 wherein either the test compound or the prolyl 4-hydroxylase protein is bound to a solid support.

59. A method of screening for candidate therapeutic agents useful for treating cancer, an inflammatory disorder, or a fibrotic disease, comprising the steps of:
assaying for expression of a polynucleotide encoding a human prolyl 4-hydroxylase protein comprising the amino acid sequence of SEQ ID NO:2 or SEQ ID NO:4 in the presence and absence of a test compound; and
identifying a test compound that decreases the expression as a candidate therapeutic agent that may be useful for treating cancer, an inflammatory disorder, or a fibrotic disease and identifying a test compound that increases the expression as a candidate therapeutic agent for treating cardiovascular disorders, anemia, or stroke.

60. The method of claim 59 wherein the step of contacting is in a cell.

61. The method of claim 59 wherein the step of contacting is in a cell-free in vitro translation system.

62-68. (canceled)
